(19) United States
(12) Patent Application Publication
(10) Pub. No.: US 2016/0161500 A1
(43) Pub. Date: Jun. 9, 2016

(54) METHOD OF SCREENING AN ANTIBODY

(71) Applicant: SAMSUNG ELECTRONICS CO., LTD., Suwon-si (KR)

(72) Inventors: Saet Byoul Lee, Seoul (KR); Dongkyu Shin, Seongnam-si (KR); Kyungah Kim, Seongnam-si (KR); Jimin Lee, Suwon-si (KR)

(21) Appl. No.: 14/962,510

(22) Filed: Dec. 8, 2015

(30) Foreign Application Priority Data
Dec. 8, 2014 (KR) 10-2014-0175232

(51) Int. Cl. G01N 33/68; A61K 31/4025; C07K 16/32; A61K 47/48

(52) U.S. Cl. CPC G01N 33/6854; A61K 47/48569; A61K 31/4025; C07K 16/32; C07K 2317/76; A61K 2039/505

(57) ABSTRACT

A method of screening an antibody including measuring presence or level of an antigen-antibody binding at various pH and an antibody-drug conjugate containing the selected antibody.

### CROSS-REFERENCE TO RELATED APPLICATIONS

This application claims the benefit of Korean Patent Application No. 10-2014-0175232 filed on Dec. 8, 2014 in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 151,085 byte ASCII (Text) file named “722184 ST25.TXT,” created Dec. 8, 2015.

### BACKGROUND OF THE INVENTION

1. Field

Provided is a method of screening an antibody including measuring presence or level of an antigen-antibody binding at various pH and an antibody-drug conjugate containing the screened antibody.

2. Description of the Related Art

A technology using affinity to a target (antigen) has been generally employed for screening a therapeutic antibody. In this case, the affinity is generally measured at a fixed pH. However, in a living body, the pH of environment varies during an antibody is transferred from blood to endosome in a target cell. Although an antibody has a high affinity to an antigen at a certain pH, if the affinity to its target (antigen) varies depending on pH change (that is, the antibody is easily separated from its target at other pH in environment of a living body), the antibody cannot exhibit its therapeutic effect.

There is a need for antibodies that bind to antigens at multiple pH conditions and for a method of identifying such antibodies.

### BRIEF SUMMARY OF THE INVENTION

An embodiment provides a method of screening an antibody, the method including measuring presence or level of antigen-antibody binding at various pH. The antibody may be an antibody for inducing cell-internalization and/or intracellular degradation of the antigen. The method of screening an antibody may further include contacting the antibody with an antigen of the antibody, prior to the step of measuring, and/or selecting the antibody as an antibody for inducing cell-internalization and/or intracellular degradation of the antigen, if the antibody maintains the antigen-antibody binding at the range of pH of endosome or lysosome in a cell, after the step of measuring.

Another embodiment provides an antibody-drug conjugate (ADC) including the screened (selected) antibody.

Another embodiment provides a method of treating and/or preventing a cancer including administering the anticancer agent including the selected antibody and/or an antibody-drug conjugate including the selected antibody and an anticancer drug to a subject in need of treating and/or preventing a cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing deference in binding energies of various anti-c-Met antibodies and c-Met depending on pH change.

FIG. 2 is a graph showing a level of releasing of various anti-c-Met antibodies from c-Met in EBC-1 cell line depending on pH change, wherein the pH is changed after antigen-antibody binding.

FIG. 3 is a graph showing a level of binding between various anti-c-Met antibodies and c-Met in EBC-1 cell line depending on pH change, wherein the antibody is treated after pH change.

FIG. 4 displays immunoblotting results showing a level of degradation of c-Met by various anti-c-Met antibodies in EBC-1 cell line.

FIG. 5 is a graph a level of degradation of c-Met by various anti-c-Met antibodies in EBC-1 cell line grafted xenograft model.

FIG. 6 displays fluorescent images showing movement of an antic-Met antibody to lysosome in EBC-1 cell line.

FIG. 7 displays fluorescent images showing movement of an antic-Met antibody to lysosome in MKN45 cell line.

FIG. 8 is a graph showing anticancer effects (cancer cell proliferation inhibition effects) of various anti-c-Met antibodies in Hs746T cell line.

FIG. 9 is a graph showing anticancer effects (cancer cell proliferation inhibition effects) of various anti-c-Met antibodies in MKN45 cell line.

FIG. 10 is a graph showing changes in tumor size by various anti-c-Met antibodies in EBC-1-grafted xenograft model.

FIG. 11 is a graph showing anticancer effects (cancer cell proliferation inhibition effects) of an anti-c-Met antibody and an ADC of anti-c-Met antibody and docetaxel in EBC-1 cell line.

FIG. 12 is a graph showing anticancer effects (cancer cell proliferation inhibition effects) of an anti-c-Met antibody and an ADC of anti-c-Met antibody and docetaxel in Hs746T cell line.

FIG. 13 is a graph showing anticancer effects (cancer cell proliferation inhibition effects) of an anti-c-Met antibody and an ADC of anti-c-Met antibody and docetaxel in MKN45 cell line.

### DETAILED DESCRIPTION OF THE INVENTION

Provided is a technology for screening for a therapeutic antibody having excellent therapeutic effect by measuring presence or level of antigen-antibody binding at various pH conditions and selecting an antibody that binds its target antigen under multiple such conditions.

Generally, the pH in a living body is about 7.4. However, the pH in a living body varies in different body parts (tissues). The pH of blood or cytoplasm is usually about pH7.4, whereas the pH of stomach is maintained in a very low range and the pH of the colon is in alkalinity range. In a cell, the pH varies between cell organelles; for example, pH inside the endosome is maintained in weak acidity of about pH 6.0 or lower or about pH 6.5 or lower, and pH inside the lysosome is maintained in acidity of about pH 4.5 or lower or about pH 5.5 or lower.

An antibody that maintains the binding to its target (antigen) and is not easily released from the target even at low pH (e.g., pH4.5 or lower) may be capable of maintaining high affinity to its target and not being easily released from the target under any intracellular condition, thereby increasing its intracellular delivery efficacy and exhibiting excellent therapeutic effect. Effective intracellular degradation of the target may be achieved while reducing side effects caused by recycling of the target, as well as leading to an increased cancer cell specific anticancer effect. Thus, it is advantageous in development of therapeutic antibodies to screen such antibodies according to the methods provided herein.

An embodiment provides a method of screening an antibody having therapeutic effect and a method of preparing an antibody-drug conjugate containing the antibody. In particular, provided is a method of screening an antibody capable of cell-internalization and/or intracellular degradation of an antigen to which the antibody specifically binds, the method comprising measuring pH-dependent antibody-antigen binding.

As used herein, the term “antibody” may be any antibody or antigen binding fragment thereof without limitation. Antibody as used herein may refer to a non-naturally occurring antibody (e.g., a recombinant or synthetic antibody) as well as a naturally occurring antibody (e.g., an antibody produced in immune system by antigenic stimulation). For example, the antibody may be an antibody or an antigen-binding fragment thereof having an intact structure (e.g., a polymeric polypeptide structure with two heavy chains and two light chains) of immunoglobulin (e.g., IgA, IgD, IgG (IgG1, IgG2, IgG3, or IgG4), IgM, or IgE). The antigen-binding fragment may be selected from the group consisting of scFv, (scFv)2, scFv-Fc, Fab, Fab′, and F(ab′)2.

In order that an antibody specifically recognizes its antigen and successfully binds to the antigen, it may be advantageous that the antibody can form and/or maintain an antibody-antigen binding at general pH conditions of a living body in which the antigen locates, such as, cell surface, cytoplasm, or blood; for example, it may range pH 6.6 to 8.5, pH 6.8 to 8.3, pH 7 to 8, pH 7.2 to 7.6, pH 7.3 to 7.5, or about pH 7.4. Therefore, the method of screening an antibody may comprise or consist essentially of measuring an antibody-antigen binding at pH 6.6 to 8.5, for example, pH 6.8 to 8.3, pH 7 to 8, pH 7.2 to 7.6, pH 7.3 to 7.5, or about pH 7.4. The step of measuring an antibody-antigen binding at such pH range may be performed by measuring an antibody-antigen binding at one or more points (values) of pH selected from the above described pH range. The step of measuring an antibody-antigen binding may comprise or consist essentially of (or consist of) measuring a level of an antibody-antigen binding and/or determining whether or not an antibody-antigen binding occur (or whether or not an antibody-antigen complex is present). The level of an antibody-antigen binding may be measured by a binding affinity of the antibody to an antigen. In this case, the method of screening an antibody may comprise measuring a binding affinity of the antibody to an antigen at one or more points (values) of pH of the above range. A high binding affinity (low Kd) is indicative of an antibody that binds well under a given pH condition and can be used as a criteria for antibody selection according to the method. For example, the binding affinity (Kd value; for example, it can be measured by any conventional method such as Biacore, ELISA, and the like) of the antibody to an antigen at the above pH range may be about 10 mM or lower, about 1 mM or lower, about 1 nM or lower, or about 1 pM or lower, such as about 0.01 pM to about 10 mM, about 0.1 pM to about 10 mM, about 1 pM to about 10 mM, about 1 nM to about 10 mM, about 1 mM to about 10 mM, about 0.01 pM to about 1 mM, about 0.1 pM to about 1 mM, about 1 pM to about 1 mM, about 1 nM to about 1 mM, about 0.01 pM to about 1 nM, about 0.1 pM to about 1 nM, about 1 pM to about 1 nM, about 0.01 pM to about 1 pM, or about 0.1 pM to 1 pM.

In use, the antibody may be internalized and/or degraded in a cell together with an antigen, thereby having an antagonistic activity to the antigen. For successful internalization (into a cell) and/or intracellular degradation, a successful movement (delivery) of an antibody-antigen complex to endosome and/or lysosome may be necessary; thus, the antibody screening method may further comprise measuring an antibody-antigen binding at pH selected from pH range lower than that of general environment of a living body and more similar to that of an endosome or lysosome (e.g., lower than a point selected from pH 6.6 to 8.5, for example, pH 7 to 8, pH 7.2 to 7.6, pH 7.3 to 7.5, or about pH 7.4; i.e., lower than pH 6.6, pH 7, pH 7.2, pH 7.3, or pH 7.4).

In an embodiment, in order that an antibody can be successfully internalized into a cell together with an antigen, it may be advantageous that the antibody can maintain the binding to the antigen (antibody-antigen binding) under the pH conditions in endosome as well as at general pH condition in a living body. Therefore, the antibody may be one capable of maintaining binding to the antigen at pH range of an endosome (such as pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0) as well as under the general pH condition in a living body (such as pH 6.6 to 8.5, for example, pH 7 to 8, pH 7.2 to 7.6, pH 7.3 to 7.5, or about pH 7.4). Therefore, in an embodiment, the measuring step of the method of screening an antibody may comprise measuring antibody-antigen binding at pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0, in addition to or instead of measuring antibody-antigen binding under the general pH condition in a living body (e.g., pH 6.6 to 8.5, for example, pH 7 to 8, pH 7.2 to 7.6, pH 7.3 to 7.5, or about pH 7.4). The step of measuring an antibody-antigen binding at the above pH range may be performed by measuring an antibody-antigen binding at one or more points (values) of pH selected from the above described pH range. The step of measuring an antibody-antigen binding may comprise or consist essentially of measuring a level of an antibody-antigen binding and/or determining whether or not an antibody-antigen binding occur (or whether or not an antibody-antigen complex is present). The level of an antibody-antigen binding may be measured by a binding affinity of the antibody to an antigen; in this case, the method of screening an antibody may comprise measuring a binding affinity of the antibody to an antigen at one or more points (values) of pH of the above range. A high binding affinity (low Kd), particularly under both pH conditions, is indicative of an antibody that binds well under a given pH condition and can be used as a criteria for antibody selection according to the method. For example, the binding affinity (Kd) of the antibody to an antibody at the above pH range may be about 10mM or lower, about 1 mM or lower, about 1 nM or lower, or about 1 pM or lower, for example, about 0.01 pM to about 10 mM, about 0.1 pM to about 10 mM, about 1 pM to about 10 mM, about 1 nM to about 10 mM, about 1 mM to about 10 mM, about 0.01 pM to about 1 mM, about 0.1 pM to about 1 mM, about 1 pM to about 1 mM, about 1 nM to about 1 mM, about 0.01 pM to about 1 nM, about 0.1 pM to about 1 nM, about 1 pM to about 1 nM, about 0.01 pM to about 1 pM, or about 0.1 pM to 1 pM.

In an embodiment, for a successfully intracellular degradation of an antibody together with an antigen, it may be advantageous that the antibody can maintain the binding to the antigen (antibody-antigen binding) at the pH conditions in lysosome as well as at general pH condition in a living body. Therefore, the antibody may be one capable of maintaining binding to the antigen at pH range in lysosome, such as pH 5.5 or lower, pH 5.2 or lower, pH 5.0 or lower, pH 4.8 or lower, or pH 4.5 or lower, for example, pH 3.0 to 5.5, pH 3.0 to 5.2, pH 3.0 to 5.0, pH 3.0 to 4.8, pH 3.0 to 4.5, pH 3.5 to 5.5, pH 3.5 to 5.2, pH 3.5 to 5.0, pH 3.5 to 4.8, pH 3.5 to 4.5, pH 4.0 to 5.5, pH 4.0 to 5.2, pH 4.0 to 5.0, pH 4.0 to 4.8, or pH 4.0 to 4.5 (as well as general pH condition in a living body such as pH 6.6 to 8.5, for example, pH 7 to 8, pH 7.2 to 7.6, pH 7.3 to 7.5, or about pH 7.4). Therefore, in an embodiment, the measuring step of the method of screening an antibody may comprise measuring an antibody-antigen binding at pH 5.5 or lower, pH 5.2 or lower, pH 5.0 or lower, pH 4.8 or lower, or pH 4.5 or lower, for example, pH 3.0 to 5.5, pH 3.0 to 5.2, pH 3.0 to 5.0, pH 3.0 to 4.8, pH 3.0 to 4.5, pH 3.5 to 5.5, pH 3.5 to 5.2, pH 3.5 to 5.0, pH 3.5 to 4.8, pH 3.5 to 4.5, pH 4.0 to 5.5, pH 4.0 to 5.2, pH 4.0 to 5.0, pH 4.0 to 4.8, or pH 4.0 to 4.5, in addition to or instead of measuring antibody-antigen binding under the general pH condition of a living body (e.g., pH 6.6 to 8.5, for example, pH 7 to 8, pH 7.2 to 7.6, pH 7.3 to 7.5, or about pH 7.4) and/or that of an endosome as described above. The step of measuring an antibody-antigen binding at the above pH range may be performed by measuring an antibody-antigen binding at one or more points (values) of pH selected from the above described pH range. The step of measuring an antibody-antigen binding may comprise or consist essentially of measuring a level of an antibody-antigen binding and/or determining whether or not an antibody-antigen binding occur (or whether or not an antibody-antigen complex is present). The level of an antibody-antigen binding may be measured by a binding affinity of the antibody to an antigen; in this case, the method of screening an antibody may comprise measuring a binding affinity of the antibody to an antigen at one or more points (values) of pH of the above range. A high binding affinity (low Kd), particularly under multiple pH conditions, is indicative of an antibody that binds well under a given pH condition and can be used as a criteria for antibody selection according to the method. For example, the binding affinity (Kd) of the antibody to an antibody at the above pH range may be about 10 mM or lower, about 1 mM or lower, about 1 nM or lower, or about 1 pM or lower, for example, about 0.01 pM to about 10 mM, about 0.1 pM to about 10 mM, about 1 pM to about 10 mM, about 1 nM to about 10 mM, about 1 mM to about 10 mM, about 0.01 pM to about 1 mM, about 0.1 pM to about 1 mM, about 1 pM to about 1 mM, about 1 nM to about 1 mM, about 0.01 pM to about 1 nM, about 0.1 pM to about 1 nM, about 1 pM to about 1 nM, about 0.01 pM to about 1 pM, or about 0.1 pM to 1 pM.

In an embodiment, the method of screening an antibody may comprise or consist essentially of:

1) measuring antibody-antigen binding at pH 6.6 to 8.5, for example, pH 7 to 8, pH 7.2 to 7.6, pH 7.3 to 7.5, or about pH 7.4; and

2-1) measuring antibody-antigen binding at pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0.

The steps of 1) and 2-1) may be conducted in any order.

The method of screening an antibody may further comprise, after the measuring step, selecting an antibody that forms and/or maintains the antibody-antigen binding (e.g., having similar level of binding affinities or binding energies to an antigen) at both of:

i) pH 6.6 to 8.5, for example, pH 7 to 8, pH 7.2 to 7.6, pH 7.3 to 7.5, or about pH 7.4; and

ii-1) pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0.

In another embodiment, the method of screening an antibody may comprise or consist essentially of:

1) measuring an antibody-antigen binding at pH 6.6 to 8.5, for example, pH 7 to 8, pH 7.2 to 7.6, pH 7.3 to 7.5, or about pH 7.4; and

2-2) measuring an antibody-antigen binding at pH 5.5 or lower, pH 5.2 or lower, pH 5.0 or lower, pH 4.8 or lower, or pH 4.5 or lower, for example, pH 3.0 to 5.5, pH 3.0 to 5.2, pH 3.0 to 5.0, pH 3.0 to 4.8, pH 3.0 to 4.5, pH 3.5 to 5.5, pH 3.5 to 5.2, pH 3.5 to 5.0, pH 3.5 to 4.8, pH 3.5 to 4.5, pH 4.0 to 5.5, pH 4.0 to 5.2, pH 4.0 to 5.0, pH 4.0 to 4.8, or pH 4.0 to 4.5.

The steps of 1) and 2-2) may be conducted in any order.

The method of screening an antibody may further comprise, after the measuring step, selecting an antibody that forms and/or maintains the antibody-antigen binding (e.g., maintains formation and/or level of the binding to an antigen) at both of:

i) pH 6.6 to 8.5, for example, pH 7 to 8, pH 7.2 to 7.6, pH 7.3 to 7.5, or about pH 7.4; and

ii-2) pH 5.5 or lower, pH 5.2 or lower, pH 5.0 or lower, pH 4.8 or lower, or pH 4.5 or lower, for example, pH 3.0 to 5.5, pH 3.0 to 5.2, pH 3.0 to 5.0, pH 3.0 to 4.8, pH 3.0 to 4.5, pH 3.5 to 5.5, pH 3.5 to 5.2, pH 3.5 to 5.0, pH 3.5 to 4.8, pH 3.5 to 4.5, pH 4.0 to 5.5, pH 4.0 to 5.2, pH 4.0 to 5.0, pH 4.0 to 4.8, or pH 4.0 to 4.5.

In another embodiment, the method of screening an antibody may comprise or consist essentially of:

1) measuring an antibody-antigen binding at pH 6.6 to 8.5, for example, pH 7 to 8, pH 7.2 to 7.6, pH 7.3 to 7.5, or about pH 7.4;

2-1) measuring an antibody-antigen binding at pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0; and

2-2) measuring an antibody-antigen binding at pH 5.5 or lower, pH 5.2 or lower, pH 5.0 or lower, pH 4.8 or lower, or pH 4.5 or lower, for example, pH 3.0 to 5.5, pH 3.0 to 5.2, pH 3.0 to 5.0, pH 3.0 to 4.8, pH 3.0 to 4.5, pH 3.5 to 5.5, pH 3.5 to 5.2, pH 3.5 to 5.0, pH 3.5 to 4.8, pH 3.5 to 4.5, pH 4.0 to 5.5, pH 4.0 to 5.2, pH 4.0 to 5.0, pH 4.0 to 4.8, or pH 4.0 to 4.5.

The steps of 1), 2-1), and 2-2) may be conducted in any order.

In this case, the pH of steps 1), 2-1) and/or 2-2) may be different from each other; for example, the pH of step 2-2) may be lower than the pH of step 2-1, and the pH of both steps 2-1 and 2-1 may be lower than the pH or step 1)).

The method of screening an antibody may further comprise, after the measuring step, selecting an antibody that forms and/or maintains the antibody-antigen binding (e.g., maintains formation and/or level of the binding to an antigen) at all conditions of:

i) pH 6.6 to 8.5, for example, pH 7 to 8, pH 7.2 to 7.6, pH 7.3 to 7.5, or about pH 7.4;

ii-1) pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0; and

ii-2) pH 5.5 or lower, pH 5.2 or lower, pH 5.0 or lower, pH 4.8 or lower, or pH 4.5 or lower, for example, pH 3.0 to 5.5, pH 3.0 to 5.2, pH 3.0 to 5.0, pH 3.0 to 4.8, pH 3.0 to 4.5, pH 3.5 to 5.5, pH 3.5 to 5.2, pH 3.5 to 5.0, pH 3.5 to 4.8, pH 3.5 to 4.5, pH 4.0 to 5.5, pH 4.0 to 5.2, pH 4.0 to 5.0, pH 4.0 to 4.8, or pH 4.0 to 4.5.

The antibody that forms and/or maintains the antibody-antigen binding (e.g., maintains formation and/or level of the binding to an antigen) may be:

an antibody that forms an antibody-antigen complex at the above described pH condition(s),

an antibody that has a binding affinity of 10 mM or lower, 1 mM or lower, 1 nM or lower or 1 pM or lower, 0.01 pM to 10 mM, 0.1 pM to 10 mM, 1 pM to 10 mM, 1 nM to 10 mM, 1 mM to 10 mM, 0.01 pM to 1 mM, 0.1 pM to 1 mM, 1 pM to 1 mM, 1 nM to 1 mM, 0.01 pM to 1 nM, 0.1 pM to 1 nM, 1 pM to 1 nM, 0.01 pM to 1 pM, or 0.1 pM to 1 pM, under the above described pH condition(s), or

a combination thereof. The binding affinity used as a criteria may be different for each step.

As described above, the screening (selection) results obtained by the above described methods of screening an antibody may differ depending on pH conditions, and thus, hereinafter, the “method of screening an antibody” may be exchangeable with “pH-dependent antibody screening method”.

The presence (or formation) of an antibody-antigen complex may be determined by any conventional protein detection method; for example, it may be determined by detecting a conventional enzymatic reaction, fluorescence, luminescence, and/or radiation. For example, the determination of presence (or formation) of an antibody-antigen complex may be performed determined by at least one selected from the group consisting of immunochromatography, immunohistochemistry, immunohistochemical staining, enzyme linked immunosorbent assay (ELISA), radioimmunoassay (RIA), enzyme immunoassay (EIA), fluorescence immunoassay (FIA), luminescence immunoassay (LIA), western blotting, surface plasmon resonance (SPR), microarray, flow cytometry assay, calculation of binding energy based on three-dimensional structure of an antibody and an antigen, and the like, but not be limited thereto.

In an embodiment, the presence (or formation) of an antibody-antigen complex may be determined by calculation of binding energy based on three-dimensional structure of an antibody and an antigen, for example, calculation of antibody-antigen binding strength by modeling of antibody and antigen.

A pH-dependent binding between an antibody and an antigen (which is interchangeable with the term “pH-dependent antibody-antigen binding”, which refers to antibody-antigen binding wherein the level (or strength) of the binding defers depending on pH conditions) may be predicted (estimated) by calculating binding energy using three-dimensional structure of an antibody of interest and its antigen. For calculation of binding energy, three-dimensional structure of an antibody and antigen and binding structure (or configuration) between of the antibody and antigen may be necessary.

For example, the three-dimensional structure may be obtained by the following processes:

1) X-ray crystallography: A protein (“target protein”) to be analyzed for its three-dimensional structure was cloned, overexpressed, and purified to crystallize the protein, and an electron density map of atoms consisting of the protein is obtained from the results of x-ray diffraction test for the crystalized protein and the coordinates of atoms consisting of the protein are obtained by mapping and refinement of amino acids of the protein.

2) searching Protein Data Bank (PDB; http://www.pdb.org): Most of publicly known three-dimensional structures of proteins are stored in PDB, and thus, a three-dimensional structure (or coordinates of atoms) of a target protein can be obtained by searching PDB using a name or amino acid sequence of the protein, if a three-dimensional structural information of the protein is present in the PDB.

3) Homology modeling: If a three-dimensional structure of a target protein cannot be obtainable by X-ray crystallography or PDB searching, homology modeling can be employed. The homology modeling also known as comparative modeling of protein, refers to constructing an atomic-resolution model of the “target” protein from its amino acid sequence and an experimental three-dimensional structure of a related homologous protein (the “template”). For example, the homology modeling can be conducted by BLAST (Basic Local Alignment Search Tool) searching (http://blast.ncbi.nlm.nih.gov) using the amino acid sequence of the target protein, to establish a template using at least one amino acid sequence of a protein similar to that of the target protein, and alignment of the amino acid sequences of the template and the target protein. The three-dimensional structure of the target protein can be obtained by matching the amino acid sequence of the target protein to the amino acid sequence of the template, to form a structure, thereby obtaining three-dimensional coordinates of atoms of the target protein, and optimizing the obtained three-dimensional coordinates using molecular dynamics.

The three-dimensional binding configuration may be obtained by following methods:

a) X-ray crystallography: A three-dimensional binding configuration of target proteins can be obtained by cloning, overexpressing and purifying the target proteins, then co-crystallizing the proteins to obtain crystals thereof, and then conducting a x-ray diffraction test, wherein this method can refer to the processes of 1) described above.

b) PDB searching: If a three-dimensional binding configuration of target proteins is available from PDB, three-dimensional coordinates of atoms can be obtained therefrom.

c) Protein-protein docking: Protein-protein docking is a computational modeling of the three-dimensional structure of complexes formed by interacting between two or more biological macromolecules such as proteins. Protein-protein complexes are the most commonly attempted targets of such modeling. A three-dimensional binding configuration between an antibody and antigen, whose three-dimensional structures cannot be experimentally obtainable, can be computationally predicted by the protein-protein docking. Assuming that three-dimensional structures of target proteins (for example, antibody and antigen) are rigid bodies, all the possible binding modes are established by docking a ligand (e.g., antigen) to the binding site of the target protein(e.g., an antibody), wherein the binding site of the protein is known or suggested by mutagenic or phylogenetic evidence, and then, estimated by shape complementarity, desolvation energy, and electrostatic energy of binding surfaces thereof. Among the binding modes, ones having high estimate score are selected. After making exclusions based on prior knowledge or stereochemical clash, the complex structures are optimized and re-estimated by re-calculating van der Waals energy, desolvation energy and electrostatic energy, to provide a binding configuration having low energies (Chen, R.; Weng, Z. P. Docking unbound proteins using shape complementarity, desolvation, and electrostatics. Proteins 2002, 47(3), 281-294).

In an embodiment, the binding energy (ΔG_{bind}) between two proteins is the sum of van der Waals binding energy (E_{vdw}), electrostatic binding energy between protein-protein and protein-solvent (ΔGₑₗ), experimental entropy of protein (ΔGₑₙₜᵣ), and non-polar desolvation energy (ΔGₙₚ), and can be calculated by the fallowing formula:

ΔG_{bind}=a·E_{vdw}+b·ΔGₑₗ+c·ΔGₑₙₜᵣ+d·ΔGₙₚ

(a, b, c, d: experimental weight coefficient)

From the above formula, pH-dependent binding energy ΔG_{bind}(pH) is calculated by the following formula:

ΔG_{bind}(pH)=a·E_{vdw}+b·ΔGₑₗ(pH)+c·ΔGₑₙₜᵣ+d·ΔGₙₚ

A difference between binding energies at pH 7.4 and a given pH (e.g., other than pH 7.4) is expressed ‘ΔΔG’, and calculated by the fallowing formula:

ΔΔG=ΔG_{bind}(pH)−ΔG_{bind}(pH=7.4)=b·(ΔGₑₗ(pH)−ΔGₑₗ(pH=7.4))

In the above formula for ΔΔG, b is 1 as experimentally obtained, and ΔGₑₗ(pH) is calculated by the fallowing formula:

ΔGₑₗ(pH)=ΔGₑₗ(∞)−ln(10)RT ∫_{pH}^{∞}Q(pH)dpH

Q(pH)=Σᵢ^{N}θᵢ(pH)

ΔGₑₗ(∞) is electrostatic energy at deprotonated states, θᵢ(pH) is fractional protonation of an amino acid at i^{th} position at a given pH, and ΔGₑₗ is calculated by the following formula:

$$Δ {G}_{el} = 332 {\sum }_{i} {\sum }_{j > i} \frac{{q}_{i} {q}_{j}}{{ɛ}_{m} {r}_{ij}} - 166 ( \frac{1}{{ɛ}_{m}} - \frac{1}{{ɛ}_{slv}} ) {\sum }_{i} {\sum }_{j} \frac{{q}_{i} {q}_{j}}{\sqrt{{r}_{i , j}^{2} + {α}_{i} {α}_{j} exp ( \frac{- {r}_{ij}^{2}}{4 {α}_{i} {α}_{j}} )}}$$

qᵢ and qⱼ are i^{th} and j^{th} atomic charges, respectively; αᵢ and αⱼ are effective Born radiuses of i^{th} and j^{th} atoms, respectively; r_{i,j} is distance between nuclei of i^{th} and j^{th} atoms; and εₘ and εₛₗᵥ are dielectric constant of protein and solvent, respectively (Spassov, V. Z.; Yan, L. pH-Selective mutagenesis of protein-protein interfaces: In silico design of therapeutic antibodies with prolonged half-life PROTEINS: Structure, Function, Bioinformatics 2013, 81, 704-714).

Receptor tyrosine kinases (RTKs) and various ligands thereof participate in tumorigenesis and progression of cancer. Therefore, the antibody screened or capable of being screened by the above described pH-dependent antibody screening method may be a therapeutic antibody against various disease such as a cancer, which is antagonistic to the receptor tyrosine kinase and/or ligand by specifically recognizing/or binding to the receptor tyrosine kinase and/or ligand as an antigen and inducing cell-internalization and/or intracellular degradation thereof.

For example, the receptor tyrosine kinase may be at least one selected from the group consisting of c-Met proteins, c-Met protein mutants, epidermal growth factor receptor (EGFR; ErbB1), human epidermal growth factor receptor 2 protein (HER2; ErbB2), human epidermal growth factor receptor 3 protein (HER3; ErbB3), platelet-derived growth factor receptors (PDGFR), vascular endothelial growth factor receptors (VEGFR), insulin-like growth factor 1 receptor (IGF1R), ephrin receptors, and the like, but not be limited thereto. The ligand may be one corresponding to the above receptor tyrosine kinase; for example, it may be selected from the group consisting of EGF, VEGF, PDGF, FGF, Ang2m and the like, but not be limited thereto.

For example, c-Met is a representative receptor tyrosine kinase present on cell surface. c-Met binds to its ligand, hepatocyte growth factor (HGF), to promote intracellular signal transduction, thereby stimulating cell growth, and it is overexpressed in many cancer cells, thereby widely relating to cancer occurrence, cancer metastasis, cancer cell migration, cancer cell invasion, and angiogenesis. For these reasons, tumorigenesis of cancer cell can be inhibited merely by targeting and degrading c-Met. When a c-Met-targeting antibody binds to c-Met and then moves into a cell, the movement (delivery) of antibody-antigen complex into a cell may be made via endosome having lower pH than blood. In this case, if the antibody fails to maintain the binding to antigen (c-Met) at the pH condition in endosome, thereby being released from c-Met, c-Met cannot be delivered to lysosome and may be recycled. However, if the antibody maintains the binding to antigen (c-Met) at the pH condition in endosome, thereby not being released from antigen (c-Met), antigen (c-Met) can be successfully delivered to lysosome whereby the chance of antigen (c-Met) degradation can be increased. In addition, if the antibody is capable of maintaining the binding to antigen (c-Met) thereby not being released from the antigen (c-Met) even at the pH condition in lysosome which is lower than that of endosome, the chance of antigen (c-Met) degradation can be more increased.

Therefore, the antibody screened (selected) by the pH-dependent antibody screening method any be an anti c-Met antibody or an antigen-binding fragment thereof.

The anti-c-Met antibody or an antigen-binding fragment thereof may be any type of antibody or antigen-binding fragment thereof, which is capable of specifically recognizing and/binding to c-Met. The antigen-binding fragment may be scFv, (scFv)2, scFv-Fc, Fab, Fab′ or F(ab′)2.

In an embodiment, the anti-c-Met antibody may be any antibody or antigen-binding fragment that acts on c-Met to induce intracellular internalization and degradation of c-Met. The anti-c-Met antibody may be any one recognizing a specific region of c-Met, e.g., a specific region in the SEMA domain, as an epitope.

The “c-Met” or “c-Met proteins” refer to receptor tyrosine kinases that bind to hepatocyte growth factors (HGF). The c-Met proteins may be those derived from all kinds of species, particularly a mammal, for example, those derived from a primate such as human c-Met (e.g. NP_000236.2), monkey c-Met (e.g., Macaca mulatta, NP_001162100), and the like, or those derived from a rodent such as mouse c-Met (e.g., NP_032617.2), rat c-Met (e.g., NP_113705.1), and the like. These proteins may include, for example, polypeptides encoded by the nucleotide sequence identified as GenBank Accession Number NM_000245.2, or proteins encoded by the polypeptide sequence identified as GenBank Accession Number NM_000236.2, or extracellular domains thereof. The receptor tyrosine kinase c-Met is involved in several mechanisms including cancer incidence, cancer metastasis, cancer cell migration, cancer cell penetration, angiogenesis, etc.

c-Met, a receptor for hepatocyte growth factor (HGF), may be divided into three portions: extracellular, transmembrane, and intracellular. The extracellular portion is composed of an α-subunit and a β-subunit which are linked to each other through a disulfide bond, and contains a SEMA domain responsible for binding HGF, a PSI domain (plexin-semaphorin-integrin homology domain) and an IPT domain (immunoglobulin-like fold shared by plexins and transcriptional factors domain). The SEMA domain of c-Met protein may have the amino acid sequence of SEQ ID NO: 79, and is an extracellular domain that functions to bind HGF. A specific region of the SEMA domain, that is, a region having the amino acid sequence of SEQ ID NO: 71, which corresponds to a range from amino acid residues 106 to 124 of the amino acid sequence of the SEMA domain (SEQ ID NO: 79) of c-Met protein, is a loop region between the second and the third propellers within the epitopes of the SEMA domain. The region acts as an epitope for the specific anti-c-Met antibody of the present invention.

The term “epitope” as used herein, refers to an antigenic determinant, a part of an antigen recognized by an antibody. In one embodiment, the epitope may be a region including about 5 or more contiguous amino acid residues within the SEMA domain (SEQ ID NO: 79) of c-Met protein, for instance, about 5 to about 19 contiguous amino acid residues within the amino acid sequence of SEQ ID NO: 71. For example, the epitope may be a polypeptide having about 5 to about 19 contiguous amino acids within SEQ ID NO: 71, wherein the polypeptide essentially includes the amino sequence of SEQ ID NO: 73 (EEPSQ) serving as an essential element for the epitope. For example, the epitope may be a polypeptide including, consisting essentially of, or consisting of the amino acid sequence of SEQ ID NO: 71, SEQ ID NO: 72, or SEQ ID NO: 73.

The epitope including the amino acid sequence of SEQ ID NO: 72 corresponds to the outermost part of the loop between the second and third propellers within the SEMA domain of a c-Met protein. The epitope including the amino acid sequence of SEQ ID NO: 73 is a site to which the antibody or antigen-binding fragment according to one embodiment most specifically binds.

Thus, the anti-c-Met antibody may specifically bind to an epitope which has about 5 to about 19 contiguous amino acids selected from among partial combinations of the amino acid sequence of SEQ ID NO: 71, including SEQ ID NO: 73 as an essential element. For example, the anti-c-Met antibody may specifically bind to an epitope including the amino acid sequence of SEQ ID NO: 71, SEQ ID NO: 72, or SEQ ID NO: 73.

In one embodiment, the anti-c-Met antibody or an antigen-binding fragment thereof may comprise or consist essentially of:

at least one heavy chain complementarity determining region (CDR) selected from the group consisting of (a) a CDR-H1 including the amino acid sequence of SEQ ID NO: 4; (b) a CDR-H2 including the amino acid sequence of SEQ ID NO: 5, SEQ ID NO: 2, or an amino acid sequence having about 8-19 consecutive amino acids within SEQ ID NO: 2 including amino acid residues from the 3^{rd} to 10^{th} positions of SEQ ID NO: 2; and (c) a CDR-H3 including the amino acid sequence of SEQ ID NO: 6, SEQ ID NO: 85, or an amino acid sequence having about 6-13 consecutive amino acids within SEQ ID NO: 85 including amino acid residues from the 1^{st} to 6^{th} positions of SEQ ID NO: 85, or a heavy chain variable region including the at least one heavy chain complementarity determining region;

at least one light chain complementarity determining region (CDR) selected from the group consisting of (a) a CDR-L1 including the amino acid sequence of SEQ ID NO: 7, (b) a CDR-L2 including the amino acid sequence of SEQ ID NO: 8, and (c) a CDR-L3 including the amino acid sequence of SEQ ID NO: 9, SEQ ID NO: 15, SEQ ID NO: 86, or an amino acid sequence having 9-17 consecutive amino acids within SEQ ID NO: 89 including amino acid residues from the 1^{st} to 9^{th} positions of SEQ ID NO: 89, or a light chain variable region including the at least one light chain complementarity determining region;

a combination of the at least one heavy chain complementarity determining region and at least one light chain complementarity determining region; .or

a combination of the heavy chain variable region and the light chain variable region.

Herein, the amino acid sequences of SEQ ID NOS: 4 to 9 are respectively represented by following Formulas Ito VI, below:

Xaa₁-Xaa₂-Tyr-Tyr-Met-Ser (SEQ ID NO: 4), Formula I

wherein Xaa₁ is absent or Pro or Ser, and Xaa₂ is Glu or Asp,

Arg-Asn-Xaa₃-Xaa₄-Asn-Gly-Xaa₅-Thr (SEQ ID NO: 5), Formula II

wherein Xaa₃ is Asn or Lys, Xaa₄ is Ala or Val, and Xaa₅ is Asn or Thr,

Asp-Asn-Trp-Leu-Xaa₆-Tyr (SEQ ID NO: 6), Formula III

wherein Xaa₆ is Ser or Thr,

Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7) Formula IV

wherein Xaa₇ is His, Arg, Gln, or Lys, Xaa₈ is Ser or Trp, Xaa₉ is His or Gln, and Xaa₁₀ is Lys or Asn,

Trp-Xaa₁₁-Ser-Xaa₁₂-Arg-Val-Xaa₁₃(SEQ ID NO: 8) Formula V

wherein Xaa₁₁ is Ala or Gly, Xaa₁₂ is Thr or Lys, and Xaa₁₃ is Ser or Pro, and

Xaa₁₄-Gln-Ser-Tyr-Ser-Xaa₁₅-Pro-Xaa₁₆-Thr (SEQ ID NO: 9) Formula VI

wherein Xaa₁₄ is Gly, Ala, or Gln, Xaa₁₅ is Arg, His, Ser, Ala, Gly, or Lys, and Xaa₁₆ is Leu, Tyr, Phe, or Met.

In one embodiment, the CDR-H1 may include an amino acid sequence selected from the group consisting of SEQ ID NOS: 1, 22, 23, and 24. The CDR-H2 may include an amino acid sequence selected from the group consisting of SEQ ID NOS: 2, 25, and 26. The CDR-H3 may include an amino acid sequence selected from the group consisting of SEQ ID NOS: 3, 27, 28, and 85.

The CDR-L1 may include an amino acid sequence selected from the group consisting of SEQ ID NOS: 10, 29, 30, 31, 32, 33, and 106. The CDR-L2 may include an amino acid sequence selected from the group consisting of SEQ ID NOS: 11, 34, 35, and 36. The CDR-L3 may include an amino acid sequence selected from the group consisting of SEQ ID NOS: 12, 13, 14, 15, 16, 37, 86, and 89.

In another embodiment, the antibody or antigen-binding fragment may comprise or consist essentially of:

a heavy chain variable region comprising a polypeptide (CDR-H1) including an amino acid sequence selected from the group consisting of SEQ ID NOS: 1, 22, 23, and 24, a polypeptide (CDR-H2) including an amino acid sequence selected from the group consisting of SEQ ID NOS: 2, 25, and 26, and a polypeptide (CDR-H3) including an amino acid sequence selected from the group consisting of SEQ ID NOS: 3, 27, 28, and 85;

a light chain variable region comprising a polypeptide (CDR-L1) including an amino acid sequence selected from the group consisting of SEQ ID NOS: 10, 29, 30, 31, 32, 33 and 106, a polypeptide (CDR-L2) including an amino acid sequence selected from the group consisting of SEQ ID NOS: 11, 34, 35, and 36, and a polypeptide (CDR-L3) including an amino acid sequence selected from the group consisting of SEQ ID NOS 12, 13, 14, 15, 16, 37, 86, and 89; or

a combination the heavy chain variable region and the light chain variable region.

In one embodiment, the anti-c-Met antibody or antigen-binding fragment may comprise or consist essentially of a heavy chain variable region comprising the amino acid sequence of SEQ ID NO: 17, 74, 87, 90, 91, 92, 93, or 94, a light chain variable region comprising the amino acid sequence of SEQ ID NO: 129, 18, 19, 20, 21, 75, 88, 95, 96, 97, 98, 99, or 107, or a combination of the heavy chain variable region and the light chain variable region.

By way of further example, the anti-c-Met antibody or the antibody fragment may include:

a heavy chain including the amino acid sequence selected from the group consisting of the amino acid sequence of SEQ ID NO: 62 (wherein the amino acid sequence from amino acid residues from the 1^{st} to 17^{th} positions is a signal peptide), the amino acid sequence from the 18^{th} to 462^{nd} positions of SEQ ID NO: 62, the amino acid sequence of SEQ ID NO: 64 (wherein the amino acid sequence from the 1^{st} to 17^{th} positions is a signal peptide), the amino acid sequence from the 18^{th} to 461^{st} positions of SEQ ID NO: 64, the amino acid sequence of SEQ ID NO: 66 (wherein the amino acid sequence from the 1^{st} to 17^{th} positions is a signal peptide), and the amino acid sequence from the 18^{th} to 460^{th} positions of SEQ ID NO: 66; and

a light chain including the amino acid sequence selected from the group consisting of the amino acid sequence of SEQ ID NO: 68 (wherein the amino acid sequence from the 1^{st} to 20^{th} positions is a signal peptide), the amino acid sequence from the 21^{st} to 240^{th} positions of SEQ ID NO: 68, the amino acid sequence of SEQ ID NO: 70 (wherein the amino acid sequence from the 1^{st} to 20^{th} positions is a signal peptide), the amino acid sequence from the 21^{st} to 240^{th} positions of SEQ ID NO: 70, and the amino acid sequence of SEQ ID NO: 108.

For example, the anti-c-Met antibody may be selected from the group consisting of:

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 68 or the amino acid sequence from the 21⁴ to 240^{th} positions of SEQ ID NO: 68;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 68 or the amino acid sequence from the 21⁴ to 240^{th} positions of SEQ ID NO: 68;

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 68 or the amino acid sequence from the 21⁴ to 240^{th} positions of SEQ ID NO: 68;

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 70 or the amino acid sequence from the 21⁴ to 240^{th} positions of SEQ ID NO: 70;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 70 or the amino acid sequence from the 21⁴ to 240^{th} positions of SEQ ID NO: 70;

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 70 or the amino acid sequence from the 21^{st} to 240^{th} positions of SEQ ID NO: 70;

an antibody including a heavy chain including the amino acid sequence of SEQ ID NO: 62 or the amino acid sequence from the 18^{th} to 462^{nd} positions of SEQ ID NO: 62 and a light chain including the amino acid sequence of SEQ ID NO: 108;

an antibody including a heavy chain including the amino acid sequence of SEQ ID NO: 64 or the amino acid sequence from the 18^{th} to 461^{st} positions of SEQ ID NO: 64 and a light chain including the amino acid sequence of SEQ ID NO: 108; and

an antibody including a heavy chain including the amino acid sequence of SEQ ID NO: 66 or the amino acid sequence from the 18^{th} to 460^{th} positions of SEQ ID NO: 66 and a light chain including the amino acid sequence of SEQ ID NO: 108.

According to an embodiment, the anti-c-Met antibody may include a heavy chain including the amino acid sequence from the 18^{th} to 460^{th} positions of SEQ ID NO: 66 and a light chain including the sequence from the 21^{st} to 240^{th} positions of SEQ ID NO: 68, or a heavy chain including the amino acid sequence from the 18^{th} to 460^{th} positions of SEQ ID NO: 66 and a light chain including the sequence of SEQ ID NO: 108.

In another embodiment, the antibody may be a multispecific antibody targeting (recognizing and/or binding to) at least two antigens, for example a bispecific antibody targeting two antigen. For example, the antibody may be a multispecific antibody (e.g., a bispecific antibody) comprising or consisting essentially of: 1) an anti-c-Met antibody or an antigen-binding fragment thereof and 2) at least one selected from the group consisting of an anti-EGFR antibody, an anti-HER2 antibody, an anti-HER3 antibody, an anti-Ang2 antibody, an anti-VEGF antibody, and the like.

In an embodiment, the antibody may be an anti-c-Met/anti-EGFR bispecific antibody comprising or consisting essentially of an anti-c-Met antibody or an antigen-binding fragment thereof and an anti-EGFR antibody or an antigen-binding fragment thereof. In this case, the c-Met antibody or an antigen-binding fragment thereof may be as described above. The anti-EGFR antibody or an antigen-binding fragment thereof may be any antibody recognizing EGFR as an antigen or an antigen-binding fragment thereof.

For example, the anti-EGFR antibody or an antigen-binding fragment thereof may comprise or consist essentially of:

at least one heavy chain complementarity determining region selected from the group consisting of CDR-H1 including the amino acid sequence of SEQ ID NO: 109, CDR-H2 including the amino acid sequence of SEQ ID NO: 110, and CDR-H3 including the amino acid sequence of SEQ ID NO: 111 or a heavy chain variable region including the at least one heavy chain complementarity determining region;

at least one light chain complementarity determining region selected from the group consisting of CDR-L1 including the amino acid sequence of SEQ ID NO: 112, CDR-L2 including the amino acid sequence of SEQ ID NO: 113, and CDR-L3 including the amino acid sequence of SEQ ID NO: 114 or a light chain variable region including the at least one light chain complementarity determining region;

a combination of the at least one heavy chain complementarity determining region and the at least one light chain complementarity determining region; or

a combination of the heavy chain variable region and the light chain variable region.

TABLE 1

| | Heavy chain CDR | | Light chain CDR |
|---|---|---|---|
| CDR-H1 | NYDMS | CDR-L1 | TGSSSNIGNNDVS |
| | (SEQ ID NO: 109) | | (SEQ ID NO: 112) |
| CDR-H2 | GISHSSGSKYYADSVKG | CDR-L2 | DDNKRPS |
| | (SEQ ID NO: 110) | | (SEQ ID NO: 113) |
| CDR-H3 | KDATPRPLKPFDY | CDR-L3 | GSWDASLNA |
| | (SEQ ID NO: 111) | | (SEQ ID NO: 114) |

For example, the anti-EGFR antibody or an antigen-binding fragment thereof may comprise or consist essentially of a heavy chain variable region including the amino acid sequence of SEQ ID NO: 115 or SEQ ID NO: 117, a light chain variable region including the amino acid sequence of SEQ ID NO: 116 or SEQ ID NO: 118, or a combination thereof.

In a particular embodiment, the anti-EGFR antibody or an antigen-binding fragment thereof may be an anti-EGFR scFv including a heavy chain variable region including the amino acid sequence of SEQ ID NO: 115 or SEQ ID NO: 117, and a light chain variable region including the amino acid sequence of SEQ ID NO: 116 or SEQ ID NO: 118. The term “scFv” may refer to a single-chain Fv that generally includes a heavy chain variable region and a light chain variable region which are linked by a covalent bond via a peptide linker or linked at the C-terminals to have a dimer structure.

<SEQ ID NO: 115: a heavy chain variable region of an anti-EGFR antibody>

EVQLLESGGGLVQPGGSLRLSCAASGFTF SNYDMSWVRQAPGKGLEWVSGISH SSGSKYYADSVKGRFTISRDNSKNTLYLQMNSLRAEDTAVYYCAKDATPRPLKPFDY WGQGTLVTVSS

(wherein the sequences marked in bold type indicate CDRs, i.e., CDR-H1, CDR-H2, and CDR-H3, in sequence)

<SEQ ID NO: 116: a light chain variable region of an anti-EGFR antibody>

QSVLTQPPSASGTPGQRVTISCTGSSSNIGNNDVSWYQQLPGTAPKLLIYDDNK RPSGVPDRF SGSKSGTSASLAISGLRSEDEADYYCGSWDASLNAYVFGGGTKLTVLG

(wherein the sequences marked in bold type indicate CDRs, i.e., CDR-L1, CDR-L2, and CDR-L3, in sequence)

<SEQ ID NO: 117: a heavy chain variable region of an anti-EGFR antibody>

EVQLLESGGGLVQPGGSLRLSCAASGFTFSNYDMSWVRQAPGKCLEWVSGISH SSGSKYYADSVKGRFTISRDNSKNTLYLQMNSLRAEDTAVYYCAKDATPRPLKPFDY WGQGTLVTVSS

(wherein the sequences marked in bold type indicate CDRs, i.e., CDR-H1, CDR-H2, and CDR-H3, in sequence)

<SEQ ID NO: 118: a light chain variable region of an anti-EGFR antibody>

QSVLTQPPSASGTPGQRVTISCTGSSSNIGNNDVSWYQQLPGTAPKLLIYDDNK RPSGVPDRF SGSKSGTSASLAISGLRSEDEADYYCGSWDASLNAYVFGCGTKLTVLG

(wherein the sequences marked in bold type indicate CDRs, i.e., CDR-L1, CDR-L2, and CDR-L3, in sequence)

Alternatively, the anti-EGFR antibody or an antigen-binding fragment thereof may be at least one selected from the group consisting of cetuximab (Erbitux), panitumumab, an anti-EGFR antibody or an antigen-binding fragment thereof comprising a heavy chain variable region of SEQ ID NO: 121, a light chain variable region of SEQ ID NO: 123, or a combination thereof, and an anti-EGFR antibody or an antigen-binding fragment thereof comprising a heavy chain variable region of SEQ ID NO: 125, a light chain variable region of SEQ ID NO: 126, or a combination thereof.

The anti-c-Met antibodies, anti-EGFR antibodies, or anti-c-Met/anti-EGFR bispecific antibodies may be mouse-derived antibodies, mouse-human chimeric antibodies, humanized antibodies, or human antibodies. The antibodies or antigen-binding fragments thereof may be isolated from a living body or non-naturally occurring. The antibodies or antigen-binding fragments thereof may be recombinant or synthetic. The antibodies may be monoclonal.

In the anti-c-Met antibody, anti-EGFR antibody, or anti-c-Met/anti-EGFR bispecific antibody, the rest portion of the light chain and the heavy chain portion excluding the CDRs, the light chain variable region, and the heavy chain variable region as defined above, for example the light chain constant region and the heavy chain constant region, may be those from any subtype of immunoglobulin (e.g., IgA, IgD, IgE, IgG (IgG1, IgG2, IgG3, IgG4), IgM, and the like).

The term “antigen-binding fragment” used herein refers to fragments of an intact immunoglobulin including portions of a polypeptide including at least one CDR or antigen-binding regions having the ability to specifically bind to the antigen. In a particular embodiment, the antigen-binding fragment may be scFv, (scFv)₂, scFvFc, Fab, Fab′, or F(ab′)₂, but is not limited thereto.

In the anti-c-Met/anti-EGFR bispecific antibody, in order to fully perform the anti-c-Met antibody's activity to mediate intracellular migration and degradation of c-Met proteins, it may be advantageous that the anti-c-Met antibody has its own intact antibody structure. In addition, in case of the EGFR binding domain such as an anti-EGFR antibody and the like, its specific recognition and binding to EGFR is important, and thus it will be fine that just an antigen-binding fragment recognizing EGFR is included in the dual inhibitor (e.g., the bispecific antibody). Therefore, the anti-c-Met/anti-EGFR bispecific antibody may be one comprising or consisting essentially of a complete form (full length) of an anti-c-Met antibody (e.g., IgG (e.g., IgG1, IgG2, IgG3, or IgG4) type antibody; comprising two heavy chains and two light chains) and an antigen binding fragment of the anti-EGFR antibody (e.g., an anti-EGFR scFv) that may be linked to C-terminus or N-terminus (e.g., C-terminus) of the anti-c-Met antibody.

In the anti-c-Met/anti-EGFR bispecific antibody, the anti-c-Met antibody or an antigen binding fragment thereof, and the anti-EGFR antibody or an antigen binding fragment thereof may be linked via a peptide linker, or they may be linked directly and without a linker. Furthermore, a heavy chain portion and a light chain portion within the antigen binding fragment, for example, a heavy chain variable region and a light chain variable region within the scFv fragment, may be linked via a peptide linker or without a linker. The peptide linker which links the anti-c-Met antibody or the antigen binding fragment thereof and the anti-EGFR antibody or the antigen binding fragment thereof, and the peptide linker which links the heavy chain portion and the light chain portion within the antigen binding fragment, may be identical or different. The peptide linker may be include about 1 to about 100 amino acid residues, particularly about 2 to about 50, and any kinds of amino acids may be included without any restrictions. The peptide linker may include for example, Gly, Asn and/or Ser residues, and also include neutral amino acids such as Thr and/or Ala. Amino acid sequences suitable for the peptide linker may be those known in the pertinent art. Meanwhile, a length of the peptide linker may be variously determined within such a limit that the functions of the fusion protein will not be affected. For instance, the peptide linker may be formed by including a total of about 1 to about 100, about 2 to about 50, or about 5 to about 25 of one or more selected from the group consisting of Gly, Asn, Ser, Thr, and Ala. In one embodiment, the peptide linker may be represented as (GGGGS)ₙ (n is an integer of about 1 to about 10, particularly an integer of about 2 to about 5).

The anti-c-Met antibody or anti-c-Met/anti-EGFR bispecific antibody or an antigen-binding fragment thereof may be one capable of forming and/or maintaining an antibody-antigen binding at the pH condition(s) of:

i) pH 6.6 to 8.5, for example, pH 7 to 8, pH 7.2 to 7.6, pH 7.3 to 7.5, or about pH 7.4; and/or

ii) pH 6.5 or lower, pH 6.2 or lower, or pH 6.0 or lower, for example, pH 5.3 to 6.5, pH 5.3 to 6.2, pH 5.3 to 6.0, pH 5.5 to 6.5, pH 5.5 to 6.2, pH 5.5 to 6.0, pH 5.8 to 6.5, pH 5.8 to 6.2, or pH 5.8 to 6.0; and/or

iii) pH 5.5 or lower, pH 5.0 or lower, pH 4.8 or lower, or pH 4.5 or lower, for example, pH 3.0 to 5.5, pH 3.0 to 5.0, pH 3.0 to 4.8, pH 3.0 to 4.5, pH 3.5 to 5.5, pH 3.5 to 5.0, pH 3.5 to 4.8, pH 3.5 to 4.5, pH 4.0 to 5.5, pH 4.0 to 5.0, pH 4.0 to 4.8, or pH 4.0 to 4.5

The antibody screened (selected) by the above described pH-dependent antibody screening method can effectively perform a cell-internalization and/or intracellular degradation of an antigen thereby exhibiting an effective antagonistic activity to the antigen, and thus it can be used as a therapeutic antibody for treating various diseases and/or symptoms, for example. In addition, the screened (selected) antibody can be used alone or in combination with other drug, for example, in a form of an antibody-drug conjugate (ADC).

Therefore, in an embodiment, the pH-dependent antibody screening method may be a method for screening an antibody for use in preparing of an antibody-drug conjugate (ADC).

In another embodiment, the antibody to be screened (selected) by the pH-dependent antibody screening method may be an antibody contained in an antibody-drug conjugate. In this case, the pH-dependent antibody screening method may be applied for selecting an antibody-drug conjugate having the above described activity.

Another embodiment provides an anticancer agent comprising an antibody screened (selected) by the pH-dependent antibody screening method, an antibody-drug conjugate containing the screened (selected) antibody, or a combination thereof, as an active ingredient.

Another embodiment provides an antibody-drug conjugate (ADC) comprising an antibody screened (selected) by the pH-dependent antibody screening method and a drug. In the antibody-drug conjugate, the antibody and drug may be linked to each other via a linker or directly without linker. The antibody-drug conjugate is characterized by being successfully delivered into endosome and/or lysosome as well as cytoplasm. The drug may be a small molecule chemical drug, i.e., a drug other than proteins or peptides such as antibodies.

An antibody-drug conjugate (ADC) is designed for increasing anticancer effect of an antibody and overcoming a limitation by specificity of chemical drug, thereby maximizing therapeutic effects and reducing side effects. Generally, in an ADC, a drug is delivered into a cancer cell by cancer cell targeting activity of a conjugated antibody, and then released from the ADC to exhibit a cancer cell specific cytotoxicity. Various ADCs having such activities are developed for minimizing side effects such as cytotoxicity to normal cells in a patient by delivering a minimized dose of a drug specifically to cancer cells.

In developing an ADC, strategies relating to an antigen, an antibody, a drug, and a linker (when the antibody and drug are conjugated via a linker) are important; among them, a selection of antibody is particularly important. It has been reported that the therapeutic effects of ADCs considerably depend on the kinds of antibodies contained thereto, even if targets, linkers and drugs are identical. Thus, it is very important to selecting an antibody for preparing an ADC. If an antibody contained in an ADC is easily released from its antigen at pH range of endosome and/or lysosome, the recycling of the ADC will be rapidly progressed, whereby intracellular residual period of the ADC and extracellular exposing time of a drug contained in the ADC will be reduced. In contrast, if an antibody contained in an ADC maintains binging to its antigen well and does not easily released from the antigen at pH range of endosome and/or lysosome, a drug contained in the ADC can be exposed or residence inside a cell until the antibody-antigen binding is broken (cleaved) and released from the ADC (i.e., the antibody) thereby exhibiting its pharmaceutical effect (e.g., anticancer effect) before (in case that a linker that link the drug to the antibody in the ADC is cleavable) or after (in case that the linker is non-cleavable) degradation of the antibody. In this case, the chance that the drug displays cytotoxic effect on a normal cell can be considerably reduced, thereby reducing side effects of anticancer drugs.

In an ADC, an antibody and drug may be linked (e.g., by a chemical bond such as a covalent bond) via a linker, and this case, the linker may be a cleavable linker that is cleaved at a desired pH range or non-cleavable linker in which pH condition does not affect cleavability thereof.

In an embodiment, the cleavable linker may be a peptide linker, a hydrazone linker, a disulfide linker, or any combination thereof.

The peptide linker may be those including any amino acids of about 1 to about 100, for example about 2 to about50, and any kinds of amino acids may be included without any restrictions. The peptide linker may include for example, Gly, Asn and/or Ser residues, and also include neutral amino acids such as Thr and/or Ala. Amino acid sequences suitable for the peptide linker may be those known in the relevant art. Meanwhile, a length of the peptide linker may be variously determined within such a limit that the functions of the fusion protein will not be affected. For instance, the peptide linker may be formed by including a total of about 1 to about 100, about 2 to about 50, or about 5 to about 25 amino acids, each of which is independently selected from the group consisting of Gly, Asn, Ser, Thr, and Ala. In one embodiment, the peptide linker may be represented as (GGGGS)ₙ (n is an integer of about 1 to about 10, particularly an integer of about 2 to about 5).

The hydrazone linker may be any organic compound having hydrazone structure (R₁R₂C═NNH₂; wherein R₁ and R₂ are independently H or an organic compound) and biodegradability. The hydrazone linker may have pH-dependent reactivity.

The disulfide linker may be any organic compound having disulfide structure (R—S—S—R′; wherein R and R′ are independently H or an organic compound) with a disulfide bond (—S—S—) and biodegradability. The disulfide linker may have pH-dependent reactivity.

If the cleavable linker is cleaved at a desired pH range, a drug in an ADC can be released from the ADC regardless of maintenance of antibody-antigen binding, to display its inherent effect.

The non-cleavable linker may be any compound having biocompatibility and non-biodegradability. For example, the non-cleavable linker may be a thioether linker, an amide linker, or any combination thereof. The thioether linker may be any organosulphur compound which is not degraded in a living body or cell and has a thioether structure (R″—S—R′″ comprising [C—S—C] bond; wherein R″ and R′″ are independently H or an organic compound). In the case of non-cleavable linker, when an antibody contained in an ADC is released from its antibody at pH range of a desired place such as cytoplasm, endosome, and/or lysosome, as described above, or the antibody and antigen are degraded in lysosome, a drug in the ADC can be released from the ADC, to display its inherent effect.

A drug contained in an ADC may be any drug having a therapeutic effect on a disease and/or condition which is desired to be treated, for example a cancer. For example, the drug may be any anticancer drug (e.g., a small molecular chemical except antibodies). For instance, the anticancer drug may be at least one selected from the group consisting of the followings, but not be limited thereto:

1) alkylating agents comprising:

i) platinum-based compounds such as cisplatin, carboplatin, oxaliplatin, and the like,

ii) nitrogen mustard-based compounds such as mechlorethamine, cyclophosphamide, ifosfamide, melphalan, chlorambucil, and the like,

iii) ethylamine-based or methylamine-based drugs such as thiotepa, altretamine, and the like,

iv) methylhydrazine derivatives such as procarbazine and the like,

v) alkylsulfonate-based drugs such as busulfan and the like,

vi) nitrosourea-based drugs such as carmustine, lomustine, and the like, and

vii) triazine-based drugs such as dacarbazine and the like;

2) antimetabolites comprising:

i) pyrimidine derivatives such as fluorouracil (5-FU), capecitabine, cytarabine, gemcitabine, and the like,

ii) folic acid derivatives such as methotrexate (MTX) and the like,

iii) purine derivatives such as mercaptopurine (6-MP) and the like;

3) natural products comprising:

i) vinca alkaloids such as vinblastine, vincristine, vinorelvine, and the like,

ii) taxanes such as paclitaxel, docetaxel, and the like,

iii) epipodophyllotoxins such as etoposide and the like,

iv) camptothecins such as topotecan, irinotecan, and the like;

4) other antibiotics and anticancer agents comprising dactinomycin, doxorubicin, daunorubicin, mitomycin, bleomycin, and the like; and

5) prednisone, 6-thioguanine; 6-TG, and the like.

Another embodiment provides a method of treating and/or preventing a cancer in a subject comprising administering the anticancer agent comprising the selected antibody as above and/or an antibody-drug conjugate comprising the selected antibody as above and an anticancer drug to the subject. The subject may be any mammal, for example, a primate such as a human and monkey, or a rodent such as a rat and a mouse, or a cell separated therefrom or a cell culture comprising the cell, but are not be limited thereto. For example, the subject may be a cancer patient or a cancer cell (e.g., a c-Met rich cell). The cancer to be treated may be any cancer treatable by the antibody and/or the anticancer drug contained in the ADC. For example, the cancer may be a solid cancer or hematological cancer and for instance, may be, but not limited to, one or more selected from the group consisting of squamous cell carcinoma, small-cell lung cancer, non-small-cell lung cancer, adenocarcinoma of the lung, squamous cell carcinoma of the lung, peritoneal carcinoma, skin cancer, melanoma in the skin or eyeball, rectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastric cancer, pancreatic cancer, glioblastoma, cervical cancer, ovarian cancer, liver cancer, bladder cancer, hepatocellular adenoma, breast cancer, colon cancer, large intestine cancer, endometrial carcinoma or uterine carcinoma, salivary gland tumor, kidney cancer, prostate cancer, vulvar cancer, thyroid cancer, head or neck cancer, brain cancer, osteosarcoma, and the like. In addition, the cancer may be one associated with overexpression of c-Met such as lung cancer (e.g., non-small cell lung cancer) or gastric cancer, but not be limited thereto.

The anticancer agent comprising the selected antibody and/or an antibody-drug conjugate comprising the selected antibody and an anticancer drug may be formulated or administered along with at least one additive selected from the group consisting of a pharmaceutically acceptable carriers, diluents, and excipients.

The pharmaceutically acceptable carrier to be included in the composition may be those commonly used for the formulation of antibodies, which may be one or more selected from the group consisting of lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil, but are not limited thereto. The pharmaceutical composition may further include one or more selected from the group consisting of a lubricant, a wetting agent, a sweetener, a flavor enhancer, an emulsifying agent, a suspension agent, and preservative.

The anticancer agent comprising the selected antibody and/or an antibody-drug conjugate comprising the selected antibody and an anticancer drug may be administered orally or parenterally. The parenteral administration may include intravenous injection, subcutaneous injection, muscular injection, intraperitoneal injection, endothelial administration, local administration, intranasal administration, intrapulmonary administration, and rectal administration. Since oral administration leads to digestion of proteins or peptides, an active ingredient in the compositions for oral administration must be coated or formulated to prevent digestion in stomach. In addition, the compositions may be administered using an optional device that enables an active substance to be delivered to target cells.

The anticancer agent comprising the selected antibody and/or an antibody-drug conjugate comprising the selected antibody and an anticancer drug may be administered at a pharmaceutically effective amount. The term “pharmaceutically effective amount” used herein refers to an amount of the active ingredient (i.e., the selected antibody and/or an anticancer drug) exhibiting effects in preventing or treating cancer, and may be properly determined in a variety of ways, depending on factors such as formulation methods, administration methods, age of patients, body weight, gender, pathologic conditions, diets, administration time, administration route, excretion speed, and reaction sensitivity.

The anticancer agent comprising the selected antibody and/or an antibody-drug conjugate comprising the selected antibody and an anticancer drug may be formulated with a pharmaceutically acceptable carrier and/or excipient into a unit or a multiple dosage form by a method easily carried out by a skilled person in the pertinent art. The dosage form may be a solution in oil or an aqueous medium, a suspension, syrup, an emulsifying solution, an extract, powder, granules, a tablet, or a capsule, and may further include a dispersing or a stabilizing agent.

The provided method of screening an antibody can be applied for developing antibodies capable of effectively degrading targets (antigens) by examining binding activities of antibodies to antigens depending on pH conditions, and the selected antibodies are useful as therapeutic antibodies. In addition, such examination of a pH-dependent antibody-antigen binding allows to develop antibodies and/or ADCs containing the antibodies, which have reduced side effects such as non-specific release in normal cells. That is, the method of screening an antibody may be used in screening (or selecting) antibodies for preparing ADCs.

### EXAMPLES

Hereafter, the present invention will be described in detail by examples.

The following examples are intended merely to illustrate the invention and are not construed to restrict the invention.

### Example 1

### Preparation of Antibodies

The following anti-c-Met antibodies were provided:

Anti-c-Met antibody A (experimental group: an antibody specifically binding to an epitope comprising 5-19 consecutive amino acids within SEQ ID NO: 71 wherein the epitope comprises SEQ ID NO: 73(EEPSQ)): an IgG2 antibody consisting of a heavy chain of SEQ ID NO: 66 and a light chain of SEQ ID NO: 68 (wherein signal sequences are removed);

Anti-c-Met antibody B (comparative group 1): an antibody consisting of a heavy chain of SEQ ID NO: 127 and a light chain of SEQ ID NO: 128:

(heavy chain)
SEQ ID NO: 127
QVQLVQSGAEVKKPGASVKVSCKASGYTFTDYYMHWVRQAPGQGLEWMGR
VNPNRRGTTYNQKFEGRVTMTTDTSTSTAYMELRSLRSDDTAVYYCARAN
WLDYWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEAAGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGK
(light chain)
SEQ ID NO: 128
DIQMTQSPSSLSASVGDRVTITCSVSSSVSSIYLHWYQQKPGKAPKLLIY
STSNLASGVPSRFSGSGSGTDFTLTISSLQPEDFATYYCQVYSGYPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGDC;

Anti-c-Met antibody C (comparative group 2): 5D5 antibody which is separated and purified from a hybridoma (American Type Culture Collection; ATCC Cat. #HB 11895);

Anti-c-Met/anti-EGFR bispecific antibody A (experimental group): a bispecific antibody wherein an EGFR scFv (wherein C-terminus of a heavy chain variable region of SEQ ID NO: 117 and N-terminus a light chain variable region of SEQ ID NO: 118 are linked to each other via a peptide linker (GGGGSGGGGSGGGGS)) is linked to C-terminus of Fc of anti-c-Met antibody A via a peptide linker (GGGGSGGGGS).

### Example 2

### Calculation of pH-Dependent Binding Energy Between Antibody-Antigen

Information of three-dimensional structure of an antigen, c-Met, was obtained from protein data bank (PDB; “www.rcsb.org/pdb/home/home.do”) (code: 4K3J, chain: B).

Information of three-dimensional structures of anti-c-Met antibodies A, B, and C was obtained by substituting the sequence information provided in Example 1 to a homology building module, Blast search & Model Antibody Framework, in Discovery Studio program (Accelrys Inc.).

Information of binding structure (or configuration) between one of anti-c-Met antibodies A, B, and C and c-Met was obtained by substituting the information of antigen (c-Met) and antibodies obtained as above to a protein-protein docking module, Dock Proteins(ZDOCK), in Discovery Studio program (Accelrys Inc.).

pH-dependent binding energy between anti-c-Met antibodies A, B, and C, and c-Met was calculated by substituting the obtained information of antigen-antibody binding structure to a Calculate Mutation Energy (Binding) module in Discovery Studio program (Accelrys Inc.) and then, calculating difference between binding energies at each pH and at standard pH 7.4 using electrostatic energy varying with pH.

The binding energy (ΔG_{bind}) between two proteins (one of anti-c-Met antibodies A, B, and C and c-Met) is the sum of van der Waals binding energy (E_{vdw}), electrostatic binding energy between protein-protein and protein-solvent (ΔGₑₗ), experimental entropy of protein (AGₑₙₜᵣ), and non-polar solvation energy (ΔGₙₚ), and can be calculated by the fallowing formula:

ΔG_{bind}=a·E_{vdw}+b·ΔGₑₗ+cΔGₑₙₜᵣ+d·ΔGₙₚ

(a, b, c, d: experimental weight coefficient)

From the above formula, pH-dependent binding energy ΔG_{bind}(pH) is calculated by the fallowing formula:

ΔG_{bind}(pH)=a·E_{vdw}+b·ΔGₑₗ(pH)+c·ΔGₑₙₜᵣ+d·ΔGₙₚ

A difference between binding energies at pH 7.4 and a given pH (other than pH 7.4) is expressed ‘ΔΔG’, and calculated by the fallowing formula:

ΔΔG=ΔG_{bind}(pH)−ΔG_{bind}(pH=7.4)=b·(ΔGₑₗ(pH)−ΔGₑₗ(pH=7.4))

(in the above formula for ΔΔG, b is 1 as experimentally obtained).

ΔGₑₗ(pH) is calculated by the fallowing formula:

ΔGₑₗ(pH)=ΔGₑₗ(∞)−ln(10)RT ∫_{pH}^{∞}Q(pH)dpH

Q(pH)=Σᵢ^{N}θᵢ(pH)

ΔGₑₗ(∞) is electrostatic energy at deprotonated states, θᵢ(pH) is fractional protonation of an amino acid at i^{th} position at a given pH, and ΔGₑₗ is calculated by the following formula:

$$Δ {G}_{el} = 332 {\sum }_{i} {\sum }_{j > i} \frac{{q}_{i} {q}_{j}}{{ɛ}_{m} {r}_{ij}} - 166 ( \frac{1}{{ɛ}_{m}} - \frac{1}{{ɛ}_{slv}} ) {\sum }_{i} {\sum }_{j} \frac{{q}_{i} {q}_{j}}{\sqrt{{r}_{i , j}^{2} + {α}_{i} {α}_{j} exp ( \frac{- {r}_{ij}^{2}}{4 {α}_{i} {α}_{j}} )}}$$

qᵢ and qⱼ are i^{th} and j^{th} atomic charges, respectively; αᵢ and αⱼ are effective Born radiuses of i^{th} and j^{th} atoms, respectively; r_{i,j} is distance between nuclei of i^{th} and j^{th} atoms; and εₘ and εₛₗᵥ are dielectric constant of protein and solvent, respectively (Spassov, V. Z.; Yan, L. pH-Selective mutagenesis of protein-protein interfaces: In silico design of therapeutic antibodies with prolonged half-life PROTEINS: Structure, Function, Bioinformatics 2013, 81, 704-714).

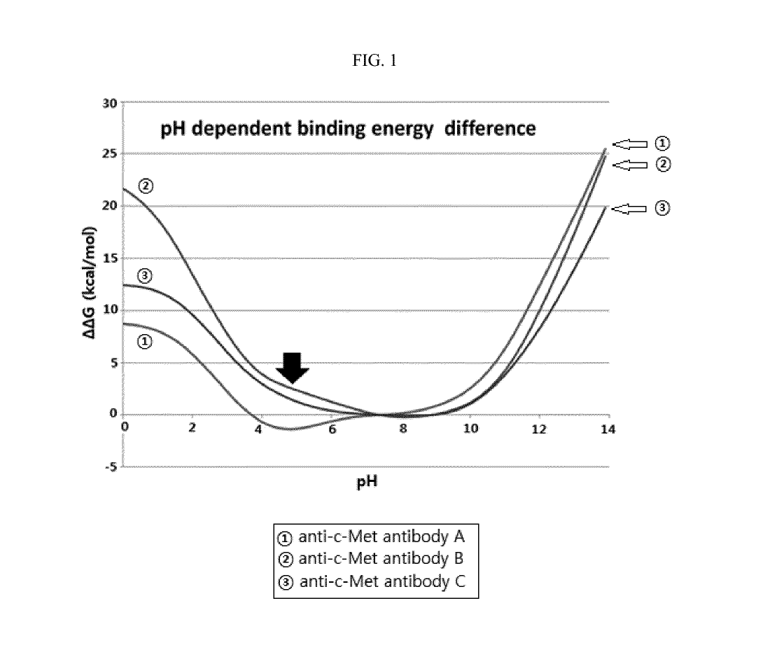

The differences between binding energies as calculated above are shown in FIG. 1 and Table 2:

TABLE 2

| | ΔΔG (c-MET antibody | ΔΔG (c-MET | ΔΔG |
|---|---|---|---|
| | A/bispecific antibody | antibody | (c-MET antibody |
| pH | A) | B) | C) |
| 4 | −0.68 | 3.89 | 2.96 |
| 4.5 | −1.26 | 2.99 | 2.01 |
| 5 | −1.32 | 2.36 | 1.28 |
| 6 | −0.62 | 1.25 | 0.40 |
| 7.4 | 0.00 | 0.00 | 0.00 |

As shown in FIG. 1 and Table 2, anti-c-Met antibody A and bispecific antibody A have relatively low binding energy with c-Met at pH7.4 or lower which is in vivo pH condition, and in particular, they have considerably low (stable) binding energy with c-Met at relatively low pH such as pH4 to pH6, compared to that of other antibodies.

### Example 3

### pH-dependent Binding of Anti-c-Met Antibody to an Antigen

In order to examine the degree of influence of pH conditions on the binding of anti-c-Met antibody and c-Met, the antibody was reacted with c-Met to form a binding therebetween, and then, treated with buffer with various pH to change the pH conditions, and then, the amount of the antibody which maintain the binding to the antigen (c-Met) at a certain pH was measured.

In particular, EBC-1 cells (ATCC) were counted and seeded in e-tubes at the amount of about 3×10⁵ cells per tube. As a medium, RPMI-1640 (Gibco) was employed. The cell culture was washed with phosphate buffer saline (PBS; pH 7.4) to remove the medium, and treated with each of c-Met antibodies A, B, and C, and c-Met bispecific antibody A, provided in Example 1, at the amount of 1 μg/ml per a tube, and then, kept in ice for 30 minutes. The obtained cells were treated with 4% formaldehyde for 10 minutes to be fixed. After washing with PBS(pH7.4), the cells treated with FACS buffer with various pH (0.2%(v/v) FBS (Fetal bovine serum) in PBS, pH 1.5, 2.0, 2.5, 3.0, 4.0, 5.0, 6.0, or 7.4) and kept in ice for 5 minutes. After washing with PBS (pH7.4), the cells were treated with 100 μl of a secondary antibody solution (Alexa488 or Alexa594-conjugated anti-human IgG antibody for c-Met antibody A and B, and bispecific antibody A; Invitrogen, 1/500 dilution in FACS buffer pH 7.4) and kept in ice for 30 minutes. The c-Met antibody attached to cell surface was quantified by Flow cytometry (BD, FACS Cantoll).

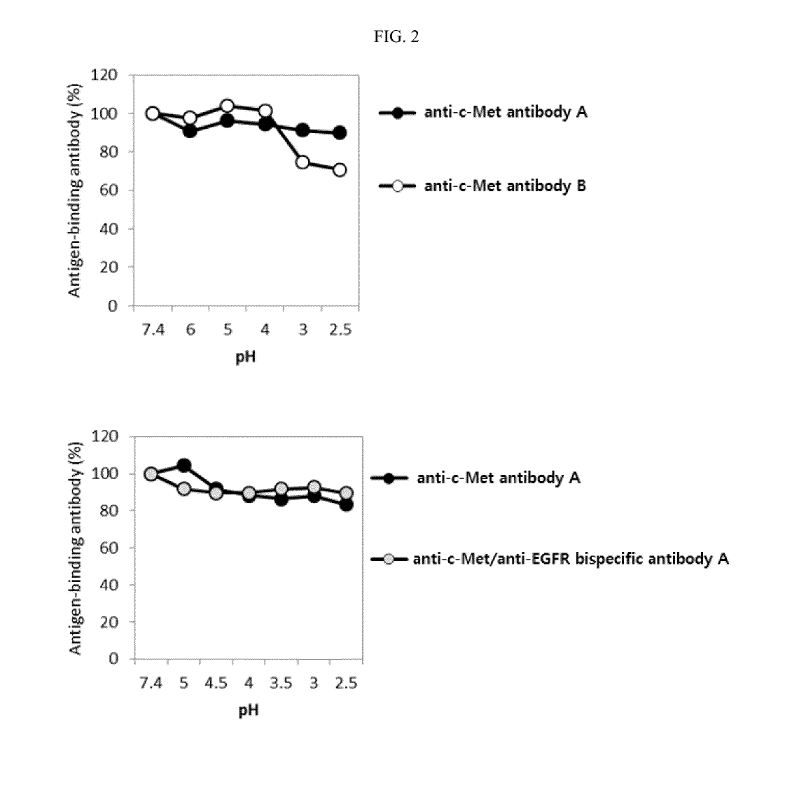

The obtained results are shown in FIG. 2. As shown in FIG. 2, when the antibody and c-Met are reacted to bind, and then treated with buffers with various pH, in case of anti-c-Met antibody A and c-Met bispecific antibody A, the binding is maintained even when buffers with relatively low pH, whereas in case of anti-c-Met antibody B, the binding is partially broken to release parts of c-Met.

In addition, an examination of the binding of an anti-c-Met antibody and c-Met present on a living cell surface with various pH conditions was performed.

In particular, EBC-1 cells (ATCC) were counted and seeded in e-tubes at the amount of about 3×10⁵ cells per tube. As a medium, RPMI-1640 (Gibco) was employed. The cell culture was washed with PBS(pH 7.4) to remove the medium, and treated with FACS buffer with various pH (0.2% (v/v) FBS in PBS, pH 1.5, 2.0, 2.5, 3.0, 4.0, 5.0, 6.0, or 7.4) and each of c-Met antibodies A, B, and C, and c-Met bispecific antibody A, provided in Example 1, at the amount of 1 μg/ml per a tube, and then, kept in ice for 30 minutes. After washing with PBS (pH7.4), the cells were treated with 100 μl of a secondary antibody solution (Alexa488 or Alexa594-conjugated anti-human IgG antibody for c-Met antibody A and B, and bispecific antibody A; Invitrogen, 1/500 dilution in FACS buffer pH 7.4) and kept in ice for 30 minutes. The c-Met antibody attached to cell surface was quantified by Flow cytometry (BD, FACS Cantoll).

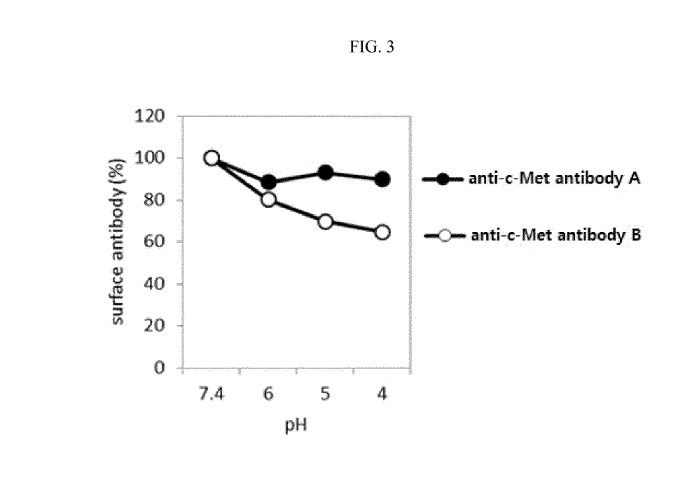

The obtained results are shown in FIG. 3. As shown in FIG. 3, anti-c-Met antibody A binds to c-Met on the cell surface well regardless of pH conditions, whereas anti-c-Met antibody B shows a lower binding rate to c-Met on the cell surface with lowering the pH conditions.

As shown in FIGS. 1 to 3, the same results were obtained using pH-dependent antibody-antigen binding modeling (FIG. 1) and the results obtained from testing actual antibodies and antigens as described above (FIGS. 2 and 3) .

### Example 4

### c-Met Degradation by Anti-c-Met Antibody

When an anti-c-Met antibody binds to c-Met and moves into cell, c-Met goes through endocytosis by endosome, wherein the pH of the endosome is known to be lower than that of serum or cytoplasm. Therefore, if an anti-c-Met antibody strongly maintains a binding to c-Met even at low pH, it may be also capable of safe delivery of c-Met into endosome or lysosome, to effectively induce internalization and degradation of c-Met.

To confirm this, c-Met degradation by each of the anti-c-Met antibodies provided in Example 1 was examined. c-Met-rich EBC-1 cells (ATCC) were treated with the anti-c-Met antibody, and then, c-Met was quantified.

In particular, EBC-1 cells were seeded in 10% (v/v) FBS supplemented RPMI-1640 medium (Gibco) at the amount of 1×10⁶ cells/well, and 24 hours after, treated with each of antibodies provided in Example 1 at the amount of 5 μg/ml per well. 30 minutes or 60 minutes after, the cells were collected and the amounts of total c-Met and phosphorylated Akt and Erk were measured by western blotting assay.

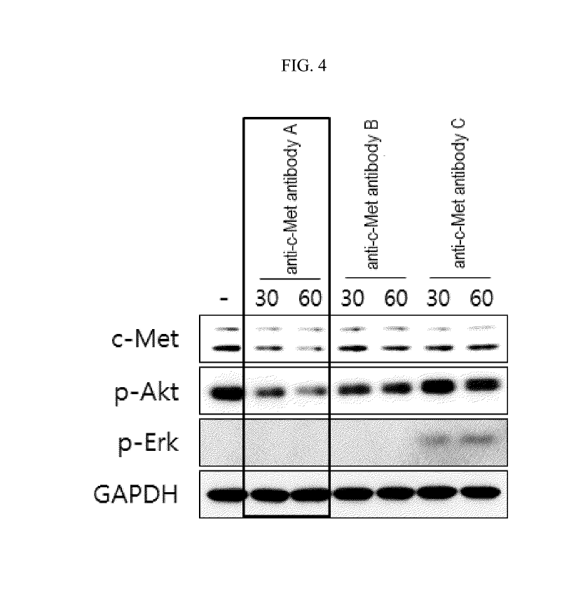

The obtained results are shown in FIG. 4. As shown in FIG. 4, c-Met antibody A exhibits excellent degradation of c-Met and inhibition of phosphorylation of downstream signal transduction substance, Akt and Erk, inside of cells, indicating that the antibody can maintain the binding to c-Met at low pH in lysosome or endosome; whereas anti-c-Met antibodies B and C exhibit relatively poor degradation of c-Met compared to c-Met antibody A, indicating that they are easily released from c-Met at low pH in lysosome or endosome.

In addition, the change in the level of c-Met in tumor tissue of EBC-1-grafted xenograft model by an anti-c-Met antibody was examined.

In particular, 5000 EBC1 cells (JCRB0820) were injected in BALB/c Nude mice. The injection was performed by making the ratio of volume of 5000 cells in serum-free media and volume of matrigel (Corning Life Sciences) to be 1:1 and the total volume thereof to be 200 μl. 7 day after the injection, when the tumor volume reaches 200 mm³, the mice were randomized and classified by allocating 15 mice per group. Each of the c-Met antibodies A and B was administered at the amount of 5 mg/kg via i.v. (intravenous) injection, once a week. 4 weeks after, the administration was performed 4 times in total and finished. After the administration, tumor tissue was excised and stored in flash frozen state for the use in protein lysis, wherein complete lysis-M (Roche, 04719956001) was used as a lysis buffer. Total amount of c-Met in 20 μg of the obtained tissue lysate was measured by sandwich ELISA using Human total HGF R/c-MET ELISA KIT (R&D systems, DYC358) according to manufacturer's manual.

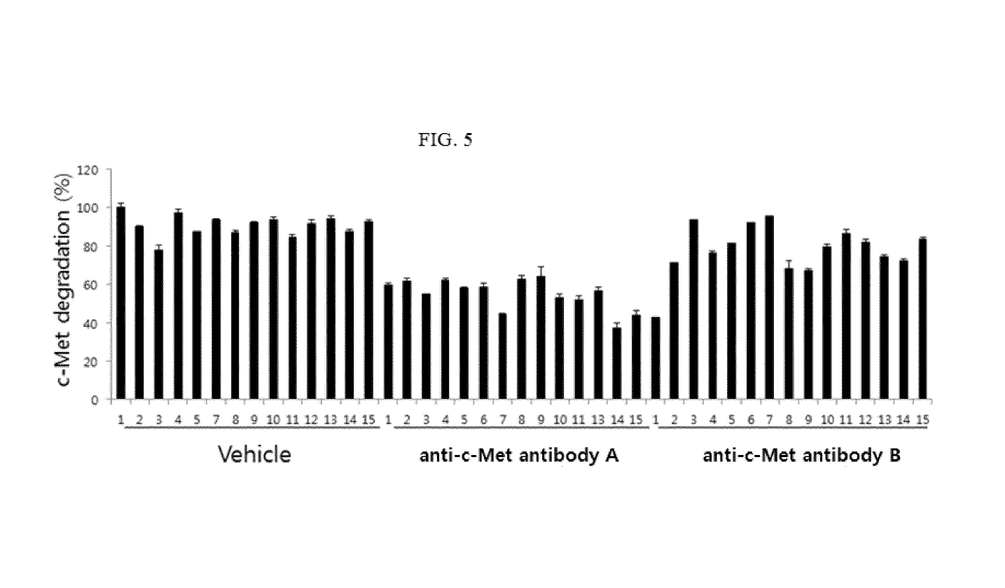

The obtained results are shown in FIG. 5. As shown in FIG. 5, c-Met antibody A can more effectively degrade c-Met compared to anti-c-Met antibody B.

### Example 5

### Lysosome Co-Localization of Anti-c-Met Antibody

To examine whether or not such excellent c-Met degradation effect of anti-c-Met antibody A is due to successful movement of the antibody together with c-Met into lysosome, co-localization of anti-c-Met antibody A and a lysosome marker was confirmed using a confocal microscope.

In particular, anti-c-Met antibody A provided in Example 1 was conjugated with Alexa Flour 647(Invitrogen) using a protein labelling kit (Invitrogen), and then, each of EBC-1 (ATCC; 20,000 cells/well) and MKN45 (JCRB (Japanese Collection of Research Bioresources, 30,000 cells/well) was treated with anti-c-Met antibody A at the amount of 1 μg/ml. 4 hours after, the cells were treated with 20 μl/well of a mixture of 1 drop of NucBlue live cell stain (Invitrogen) and 2 μl/ml of Lysotracker DND-99 (Invitrogen) in 1 ml of PBS, to stain nuclei and lysosomes, and observed by a confocal microscope (Zeiss). For obtaining fixing cell images, the cells were treated with 4% formaldehyde and kept for 10 minutes, before the treatment of the antibody.

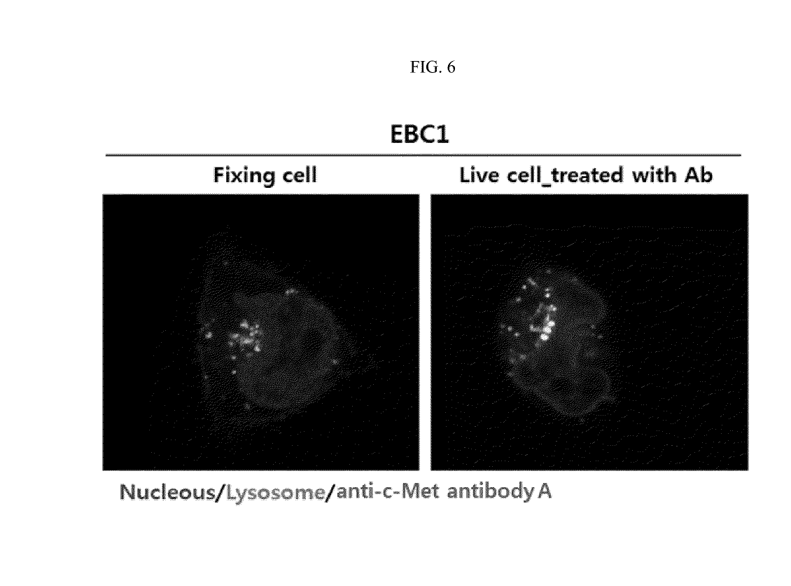

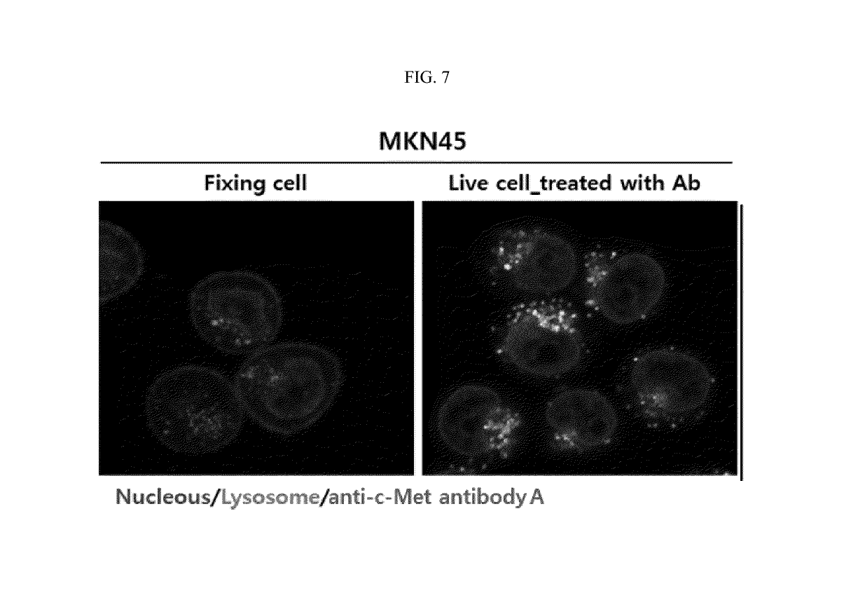

The obtained results are shown in FIG. 6 (EBC-1 cells) and FIG. 7 (MKN45 cells). In FIGS. 6 and 7, co-localization of anti-c-Met antibody A and lysosome marker (Lysotracker DND-99) as well as cell-internalization of anti-c-Met antibody are observed.

### Example 6

### Anticancer Effect of Anti-c-Met Antibody

In order to compare anticancer effects of anti-c-Met antibody A capable of maintaining the binding to c-Met even at low pH and c-Met antibody B having decreased c-Met binding ability at low pH, Hs746T cell line (JCRB) and MKN45 cell line (JCRB), in which c-Met is expressed at large amount, were treated the antibodies and then, viabilities of the above cell lines were measured.

In particular, each of Hs746T and EBC-1 cell lines was seeded onto 96-well plate, each well of which contains 10%(v/v) FBS supplemented RPMI1640 medium (GIBCO) at the amount of 5,000 cells/well, and incubated overnight at 37° C. On the next day, the cells were treated with each of the antibodies provided in Example 1 at the amount of 100 μl wherein the concentration of the antibody was serial-diluted from 10 μg/ml. After incubation for 72 hours, the cell viabilities were measured using CellTiter-GLO reagent (Promega).

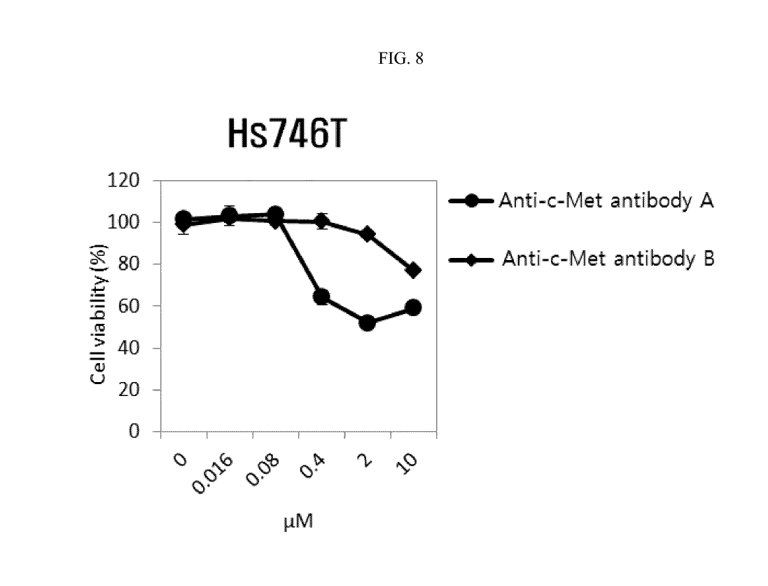

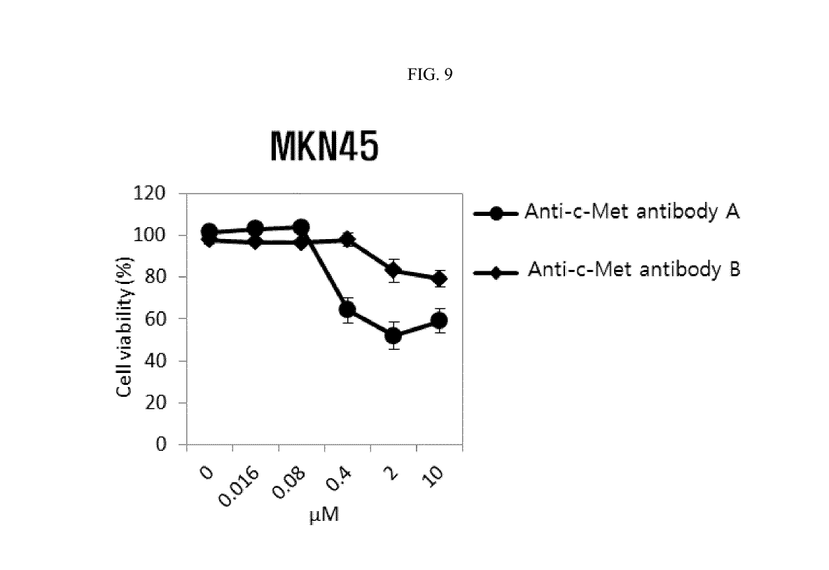

The obtained results are shown in FIG. 8 (Hs746T) and FIG. 9 (MKN45). As shown in FIGS. 8 and 9, in both cell lines, anti-c-Met antibody A capable of maintaining the binding to c-Met even at low pH has more excellent anticancer effect (cancer cell proliferation inhibition effect) compared to anti-c-Met antibody B.

In addition, the change in tumor size of an EBC-1 grafted xenograft model by treating antibodies was examined.

In particular, 5000 EBC1 cells (JCRB0820) were injected in BALB/c Nude mice. The injection was performed by making the ratio of volume of 5000 cells in serum-free media and volume of matrigel (Corning Life Sciences) to be 1:1 and the total volume thereof to be 200 μl. 7 day after the injection, when the tumor volume reaches 200 mm³, the mice were randomized and classified by allocating 15 mice per group. Each of the c-Met antibodies A, B, and C in Example 1 was administered at the amount of 5 mg/kg via i. v. injection, once a week. 4 weeks after, the administration was performed 4 times in total and finished. The volume of tumor tissue was measured on 7, 11, 16, 18, 21, 23, 25, and 28 days.

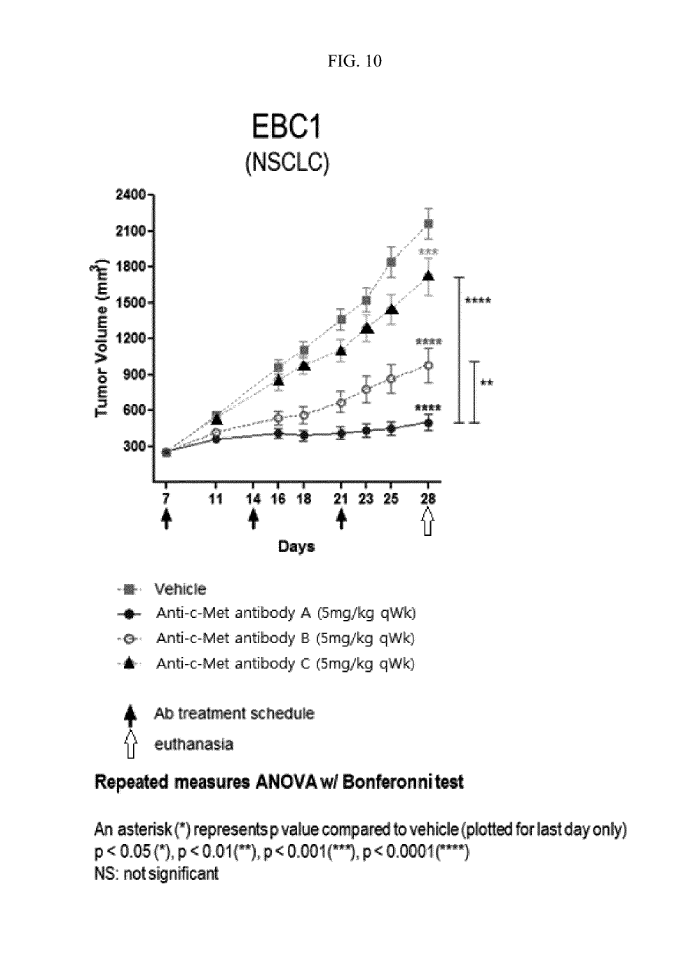

The obtained results are shown in FIG. 10. As shown in FIG. 10, anti-c-Met antibody A capable of maintaining the binding to c-Met even at low pH has more excellent anticancer effect compared to anti-c-Met antibodies B and C.

### Example 7

### Anticancer Effect of an Antibody-Drug Conjugate (ADC)

An antibody-drug conjugate (ADC) was prepared using anti-c-Met antibody A which is conformed to be relatively less pH-dependent, and the anticancer effect of the ADC was examined. For this examination, anti-c-Met antibody A prepared Example 1 and docetaxel was reacted according to the following reaction scheme, to prepared an ADC.

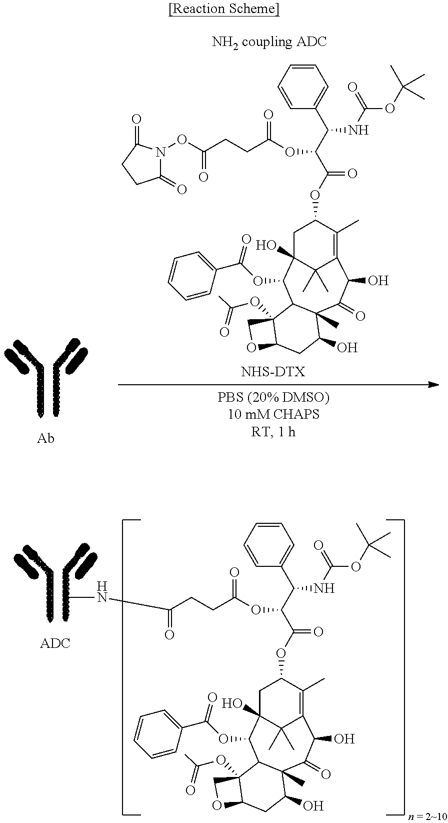

In particular, a docetaxel derivative expressed by following chemical formula 1, NHS-Docetaxel (NHS-DTX), was dissolved in DMSO (sigma) at the concentration of 5 mg/ml.

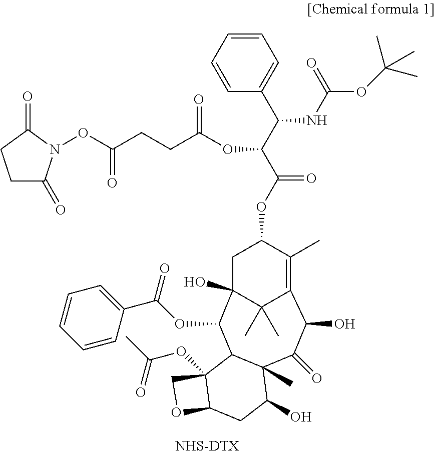 [SMILES: CC(=O)O[C@@]12CO[C@@H]1C[C@H](O)[C@@]1(C)C(=O)[C@H](O)C3=C(C)[C@@H](OC(=O)[C@H](OC(=O)CCC(=O)ON4C(=O)CCC4=O)[C@@H](NC(=O)OC(C)(C)C)c4ccccc4)C[C@@](O)([C@@H](OC(=O)c4ccccc4)C12)C3(C)C]

Anti-c-Met antibody A was provided in a solution form dissolved in 20% DMSO (Sigma), 10 mM CHAPS (Sigma), and 80% PBS (pH 7.4) (Gibco) at the final concentration of 2 mg/ml, to which the NHS-Docetaxel was added at the amount of 5-folds, 10-folds, 15-folds, or 20-folds of mole of the antibody and mixed. The mixture was kept for 1 hour at room temperature, to allow reactions therebetween. Thereafter, the antibody-docetaxel conjugate was purified using Desalting column (GE healthcare) of AKTA Prime (GE healthcare). The purification was performed by equipping desalting column in AKTA prime, flowing PBS (pH 7.4) at the fluid velocity of 5 ml/min, adding the obtained reactant into the column, and then separating the antibody-docetaxel conjugate using size difference.

The prepared ADC of anti-c-Met antibody A and docetaxel was used for the following experiment.

Each of MKN45, Hs746T and EBC-1 cell lines was seeded onto 96-well plate, each well of which contains 10%(v/v) FBS supplemented RPMI1640 medium (GIBCO) at the amount of 5,000 cells/well, and incubated overnight at 37° C. On the next day, the cells treated with prepared ADC at the amount of 100 ul/well wherein the concentration of the ADC was serial-diluted from 10 ug/ml. After incubation for 72 hours, the cell viabilities were measured using CellTiter-GLO reagent (Promega).

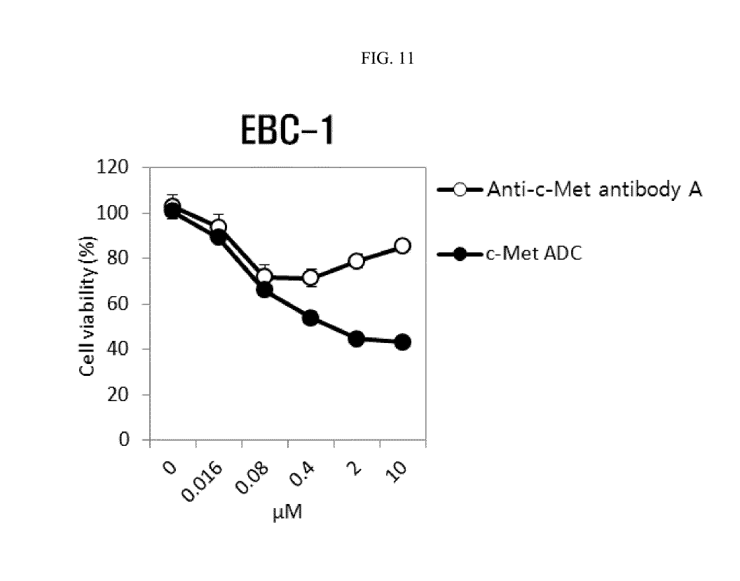

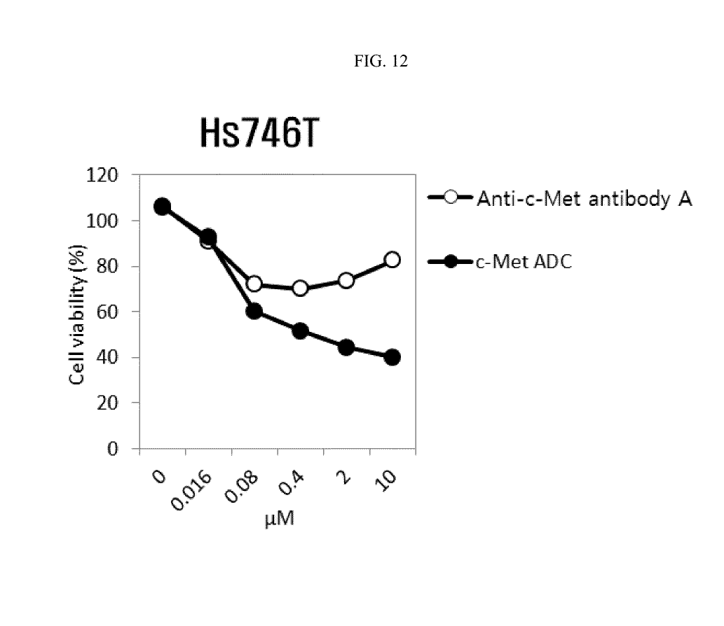

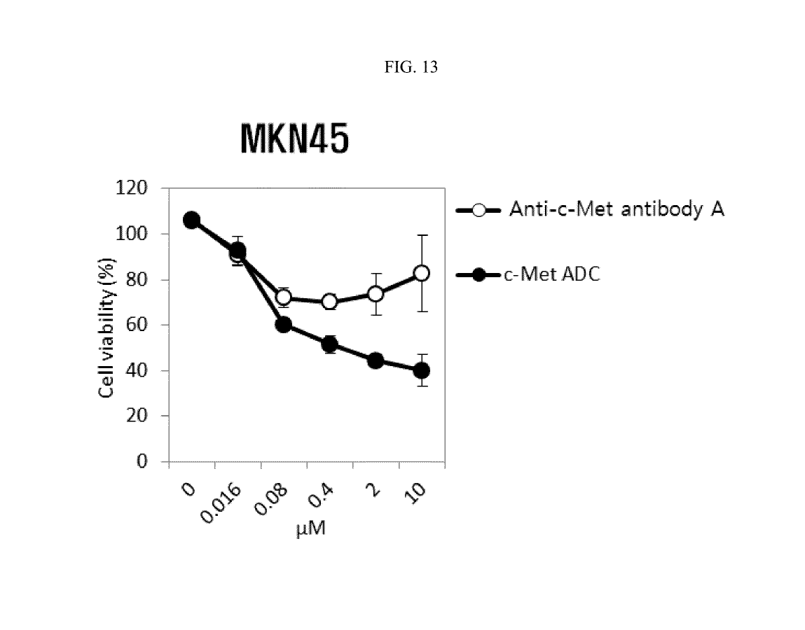

The obtained results are shown in FIG. 11 (EBC-1 cell line), FIG. 12 (Hs746T cell line), and FIG. 13 (MKN45 cell line). As shown in FIGS. 11 to 13, the ADC containing anti-c-Met antibody A capable of maintaining the binding to c-Met even at low pH has considerably synergistic anticancer effect.

What is claimed is:

1. A method of selecting an antibody comprising
1) measuring binding of the antibody to an antigen at one or more pH levels selected from pH 6.6 to 8.5,
2) measuring binding of the antibody to the antigen at one or more pH levels below pH 6.6, and
3) selecting the antibody if it binds the antigen under at least one pH level from pH 6.6 to 8.5 and at least one pH level below pH 6.6.

2. The method of claim 1, comprising
1) measuring binding of the antibody to an antigen at one or more pH levels selected from pH 6.6 to 8.5;
2-1) measuring binding of the antibody to an antigen at one or more pH levels selected from pH 6.5 or lower; and
3) selecting the antibody if it binds the antigen under at least one pH level of 1) and under at least one pH level of 2-1).

3. The method of claim 2, comprising
1) measuring binding of the antibody to an antigen at one or more pH levels selected from pH 6.6 to 8.5;
2-1′) measuring binding of the antibody to an antigen at one or more pH levels selected from pH 5.3 to 6.5; and
3) selecting the antibody if it binds the antigen under at least one pH level of 1) and under at least one pH level of 2-1′).

4. The method of claim 1, comprising
1) measuring binding of the antibody to an antigen at one or more pH levels selected from pH 6.6 to 8.5;
2-2) measuring binding of the antibody to an antigen at one or more pH levels selected from pH 5.5 or lower; and
3) selecting the antibody if it binds the antigen under at least one pH level of 1) and under at least one pH level of 2-2).

5. The method of claim 4, comprising
1) measuring binding of the antibody to an antigen at one or more pH levels selected from pH 6.6 to 8.5;
2-2′) measuring binding of the antibody to an antigen at one or more pH levels selected from pH 3.0 to 5.5; and
3) selecting the antibody if it binds the antigen under at least one pH level of 1) and under at least one pH level of 2-2′).

6. The method of claim 1, comprising
1) measuring binding of the antibody to an antigen at one or more pH levels selected from pH 6.6 to 8.5;
2-1′) measuring binding of the antibody to an antigen at one or more pH levels selected from pH 5.3 to 6.5;
2-2′) measuring binding of the antibody to an antigen at one or more pH levels selected from pH 3.0 to 5.5; and
3) selecting the antibody if it binds the antigen under at least one pH level selected in each of steps 1), 2-1′), and 2-2′),
wherein the pH levels selected in each of 2-1′) and 2-2′) are different from each other.

7. The method of claim 1, wherein the binding of the antibody to the antigen is measured by immunochromatography, immunohistochemistry, immunohistochemical staining, enzyme linked immunosorbent assay (ELISA), radioimmunoassay (RIA), enzyme immunoassay (EIA), fluorescence immunoassay (FIA), luminescence immunoassay (LIA), western blotting, surface plasmon resonance (SPR), microarray, flow cytometry assay, calculation of binding energy based on three-dimensional structure of an antibody and an antigen, or a combination thereof.

8. The method of claim 1, further comprising conjugating the selected antibody to a drug.

9. An anticancer agent comprising the antibody selected by the method of claim 1.

10. An antibody-drug conjugate comprising the antibody selected by the method of claim 1 and a drug.

11. The antibody-drug conjugate of claim 10, further comprising a cleavable linker linking the antibody and the drug.

12. The antibody-drug conjugate of claim 11, wherein the cleavable linker is a peptide linker, a hydrazone linker, a disulfide linker, or a combination thereof.

13. The antibody-drug conjugate of claim 10, further comprising a non-cleavable linker linking the antibody and the drug.

14. The antibody-drug conjugate of claim 13, wherein the non-cleavable linker is a thioether linker, an amide linker, or a combination thereof.

15. A method of treating a cancer in a subject, comprising administering the anticancer agent of claim 9 to the subject.

16. A method of treating a cancer in a subject, comprising administering the antibody-drug conjugate of claim 10.

17. The method of claim 16, wherein the antibody-drug conjugate further comprises a cleavable linker linking the antibody and the drug.

18. The method of claim 17, wherein the cleavable linker is a peptide linker, a hydrazone linker, a disulfide linker, or a combination thereof.

19. The method of claim 16, wherein the antibody-drug conjugate further comprises a non-cleavable linker linking the antibody and the drug.

20. The method of claim 19, wherein the non-cleavable linker is a thioether linker, an amide linker, or a combination thereof.


SEQUENCE LISTING (129 sequences)

SEQ ID NO: 1 - protein, 5 aa, Artificial Sequence
  Synthetic heavy chain CDR1 of AbF46
DYYMS

SEQ ID NO: 2 - protein, 19 aa, Artificial Sequence
  Synthetic heavy chain CDR2 of AbF46
FIRNKANGYTTEYSASVKG

SEQ ID NO: 3 - protein, 6 aa, Artificial Sequence
  Synthetic heavy chain CDR3 of AbF46
DNWFAY

SEQ ID NO: 4 - protein, 6 aa, Artificial Sequence
  Synthetic heavy chain CDR1 of c-Met antibody
XXYYMS

SEQ ID NO: 5 - protein, 8 aa, Artificial Sequence
  Synthetic heavy chain CDR2 of c-Met antibody
RNXXNGXT

SEQ ID NO: 6 - protein, 6 aa, Artificial Sequence
  Synthetic heavy chain CDR3 of c-Met antibody
DNWLXY

SEQ ID NO: 7 - protein, 17 aa, Artificial Sequence
  Synthetic light chain CDR1 of c-Met antibody
KSSXSLLAXGNXXNYLA

SEQ ID NO: 8 - protein, 7 aa, Artificial Sequence
  Synthetic light chain CDR2 of c-Met antibody
WXSXRVX

SEQ ID NO: 9 - protein, 9 aa, Artificial Sequence
  Synthetic light chain CDR3 of c-Met antibody
XQSYSXPXT

SEQ ID NO: 10 - protein, 17 aa, Artificial Sequence
  Synthetic light chain CDR1 of AbF46
KSSQSLLASGNQNNYLA

SEQ ID NO: 11 - protein, 7 aa, Artificial Sequence
  Synthetic light chain CDR2 of AbF46
WASTRVS

SEQ ID NO: 12 - protein, 9 aa, Artificial Sequence
  Synthetic light chain CDR3 of AbF46
QQSYSAPLT

SEQ ID NO: 13 - protein, 9 aa, Artificial Sequence
  Synthetic CDR-L3 derived from L3-1 clone
QQSYSRPYT

SEQ ID NO: 14 - protein, 9 aa, Artificial Sequence
  Synthetic CDR-L3 derived from L3-2 clone
GQSYSRPLT

SEQ ID NO: 15 - protein, 9 aa, Artificial Sequence
  Synthetic CDR-L3 derived from L3-3 clone
AQSYSHPFS

SEQ ID NO: 16 - protein, 9 aa, Artificial Sequence
  Synthetic CDR-L3 derived from L3-5 clone
QQSYSRPFT

SEQ ID NO: 17 - protein, 117 aa, Artificial Sequence
  Synthetic heavy chain variable region of anti c-Met humanized antibody(huAbF46-H4)
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSS

SEQ ID NO: 18 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKR

SEQ ID NO: 19 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCGQSYSRPLTFGQGTKVEIKR

SEQ ID NO: 20 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCAQSYSHPFSFGQGTKVEIKR

SEQ ID NO: 21 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPFTFGQGTKVEIKR

SEQ ID NO: 22 - protein, 6 aa, Artificial Sequence
  Synthetic CDR-H1 derived from H11-4 clone
PEYYMS

SEQ ID NO: 23 - protein, 6 aa, Artificial Sequence
  Synthetic CDR-H1 derived from YC151 clone
PDYYMS

SEQ ID NO: 24 - protein, 6 aa, Artificial Sequence
  Synthetic CDR-H1 derived from YC193 clone
SDYYMS

SEQ ID NO: 25 - protein, 8 aa, Artificial Sequence
  Synthetic CDR-H2 derived from YC244 clone
RNNANGNT

SEQ ID NO: 26 - protein, 8 aa, Artificial Sequence
  Synthetic CDR-H2 derived from YC321 clone
RNKVNGYT

SEQ ID NO: 27 - protein, 6 aa, Artificial Sequence
  Synthetic CDR-H3 derived from YC354 clone
DNWLSY

SEQ ID NO: 28 - protein, 6 aa, Artificial Sequence
  Synthetic CDR-H3 derived from YC374 clone
DNWLTY

SEQ ID NO: 29 - protein, 17 aa, Artificial Sequence
  Synthetic CDR-L1 derived from L1-1 clone
KSSHSLLASGNQNNYLA

SEQ ID NO: 30 - protein, 17 aa, Artificial Sequence
  Synthetic CDR-L1 derived from L1-3 clone
KSSRSLLSSGNHKNYLA

SEQ ID NO: 31 - protein, 17 aa, Artificial Sequence
  Synthetic CDR-L1 derived from L1-4 clone
KSSKSLLASGNQNNYLA

SEQ ID NO: 32 - protein, 17 aa, Artificial Sequence
  Synthetic CDR-L1 derived from L1-12 clone
KSSRSLLASGNQNNYLA

SEQ ID NO: 33 - protein, 17 aa, Artificial Sequence
  Synthetic CDR-L1 derived from L1-22 clone
KSSHSLLASGNQNNYLA

SEQ ID NO: 34 - protein, 7 aa, Artificial Sequence
  Synthetic CDR-L2 derived from L2-9 clone
WASKRVS

SEQ ID NO: 35 - protein, 7 aa, Artificial Sequence
  Synthetic CDR-L2 derived from L2-12 clone
WGSTRVS

SEQ ID NO: 36 - protein, 7 aa, Artificial Sequence
  Synthetic CDR-L2 derived from L2-16 clone
WGSTRVP

SEQ ID NO: 37 - protein, 9 aa, Artificial Sequence
  Synthetic CDR-L3 derived from L3-32 clone
QQSYSKPFT

SEQ ID NO: 38 - DNA, 1416 bp, Artificial Sequence
  Synthetic nucleotide sequence of heavy chain of chAbF46
gaattcgccgccaccatggaatggagctgggtttttctcgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatggacaccctgagagctgaggacagtgccacttattactgtgcaagagataactggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 39 - DNA, 759 bp, Artificial Sequence
  Synthetic nucleotide sequence of light chain of chAbF46
gaattcactagtgattaattcgccgccaccatggattcacaggcccaggtcctcatgttgctgctgctatcggtatctggtacctgtggagacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatgctgataatttgggcatccactagggtatctggagtccctgatcgcttcataggcagtggatctgggacggatttcactctgaccatcaacagtgtgcaggctgaagatctggctgtttattactgtcagcagtcctacagcgctccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 40 - protein, 447 aa, Artificial Sequence
  Synthetic amino acid sequence of H1-heavy
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 447 aa, Artificial Sequence
  Synthetic amino acid sequence of H3-heavy
EVQLVESGGGLVQPGGSLRLSCAASGFTFTDYYMSWVRQAPGKGLEWLGFIRNKANGYTTEYSASVKGRFTISRDNSKNSLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 447 aa, Artificial Sequence
  Synthetic amino acid sequence of H4-heavy
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 220 aa, Artificial Sequence
  Synthetic amino acid sequence of H1-light
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 220 aa, Artificial Sequence
  Synthetic amino acid sequence of H2-light
DIVMTQTPLSLPVTPGEPASISCKSSQSLLASGNQNNYLAWHLQKPGQSPQMLIIWASTRVSGVPDRFSGSGSGTDFTLKISRVEAEDVGVYYCQQSYSAPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 220 aa, Artificial Sequence
  Synthetic amino acid sequence of H3-light
DIVMTQSPDSLAVSLGERATINCKSSQSLLASGNQNNYLAWYQQKPGQPPKLLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, Artificial Sequence
  Synthetic amino acid sequence of H4-light
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 47 - DNA, 1350 bp, Artificial Sequence
  Synthetic nucleotide sequence of H1-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgaaaaccgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 48 - DNA, 1350 bp, Artificial Sequence
  Synthetic nucleotide sequence of H3-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgcgtgctgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 49 - DNA, 1350 bp, Artificial Sequence
  Synthetic nucleotide sequence of H4-heavy
gaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 50 - DNA, 669 bp, Artificial Sequence
  Synthetic nucleotide sequence of H1-light
gacatcgtgatgacccagtctccagactccctggctgtgtctctgggcgagagggccaccatcaactgcaagtccagccagagtcttttagctagcggcaaccaaaataactacttagcttggcaccagcagaaaccaggacagcctcctaagatgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 51 - DNA, 669 bp, Artificial Sequence
  Synthetic nucleotide sequence of H2-light
gatattgtgatgacccagactccactctccctgcccgtcacccctggagagccggcctccatctcctgcaagtccagtcagagtcttttagctagtggcaaccaaaataactacttggcctggcacctgcagaagccagggcagtctccacagatgctgatcatttgggcatccactagggtatctggagtcccagacaggttcagtggcagtgggtcaggcactgatttcacactgaaaatcagcagggtggaggctgaggatgttggagtttattactgccagcagtcctacagcgctccgctcacgttcggacagggtaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 52 - DNA, 669 bp, Artificial Sequence
  Synthetic nucleotide sequence of H3-light
gacatcgtgatgacccagtctccagactccctggctgtgtctctgggcgagagggccaccatcaactgcaagtccagccagagtcttttagctagcggcaaccaaaataactacttagcttggtaccagcagaaaccaggacagcctcctaagctgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 53 - DNA, 669 bp, Artificial Sequence
  Synthetic nucleotide sequence of H4-light
gatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagcgctccgctcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 54 - protein, 23 aa, Artificial Sequence
  Synthetic linker between VH and VL
GLGGLGGGGSGGGGSGGSSGVGS

SEQ ID NO: 55 - DNA, 1088 bp, Artificial Sequence
  Synthetic polynucleotide encoding scFv of huAbF46 antibody
gctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaac

SEQ ID NO: 56 - DNA, 5597 bp, Artificial Sequence
  Synthetic expression vector including polynucleotide encoding scFv of huAbF46 antibody
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 57 - protein, 13 aa, Artificial Sequence
  Synthetic U6-HC7 hinge
EPKSCDCHCPPCP

SEQ ID NO: 58 - DNA, 435 bp, Artificial Sequence
  Synthetic polynucleotide encoding CDR-L3 derived from L3-1 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacg

SEQ ID NO: 59 - DNA, 435 bp, Artificial Sequence
  Synthetic polynucleotide encoding CDR-L3 derived from L3-2 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgggcagtcctacagccgtccgctcacgttcggacagggtaccaaggtggagatcaaacgtacg

SEQ ID NO: 60 - DNA, 435 bp, Artificial Sequence
  Synthetic polynucleotide encoding CDR-L3 derived from L3-3 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgcacagtcctacagccatccgttctctttcggacagggtaccaaggtggagatcaaacgtacg

SEQ ID NO: 61 - DNA, 435 bp, Artificial Sequence
  Synthetic polynucleotide encoding CDR-L3 derived from L3-5 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtttacgttcggacagggtaccaaggtggagatcaaacgtacg

SEQ ID NO: 62 - protein, 462 aa, Artificial Sequence
  Synthetic polypeptide consisting of heavy chain of huAbF46-H4-A1, U6-HC7 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1410 bp, Artificial Sequence
  Synthetic polynucleotide encoding polypeptide consisting of heavy chain of huAbF46-H4-A1, U6-HC7 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 64 - protein, 461 aa, Artificial Sequence
  Synthetic polypeptide consisting of heavy chain of huAbF46-H4-A1, human IgG2 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1407 bp, Artificial Sequence
  Synthetic polynucleotide encoding polypeptide consisting of heavy chain of huAbF46-H4-A1, human IgG2 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagaggaagtgctgtgtggagtgccccccctgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 66 - protein, 460 aa, Artificial Sequence
  Synthetic polypeptide consisting of heavy chain of huAbF46-H4-A1, human IgG2 hinge and constant region of human IgG2
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - DNA, 1404 bp, Artificial Sequence
  Synthetic polynucleotide encoding polypeptide consisting of heavy chain of huAbF46-H4-A1, human IgG2 hinge and constant region of human IgG2
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 68 - protein, 240 aa, Artificial Sequence [sequence omitted]

SEQ ID NO: 69 - DNA, 758 bp, Artificial Sequence
  Synthetic polynucleotide encoding polypeptide consisting of light chain of huAbF46-H4-A1(H36Y) and human kappa constant region
aattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggtaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 70 - protein, 240 aa, Artificial Sequence
  Synthetic polypeptide consisting of light chain of huAbF46-H4-A1 and human kappa constant region
MDSQAQVLMLLLLSVSGTCGDIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 19 aa, Artificial Sequence
  Synthetic epitope in SEMA domain of c-Met
FSPQIEEPSQCPDCVVSAL

SEQ ID NO: 72 - protein, 10 aa, Artificial Sequence
  Synthetic epitope in SEMA domain of c-Met
PQIEEPSQCP

SEQ ID NO: 73 - protein, 5 aa, Artificial Sequence
  Synthetic epitope in SEMA domain of c-Met
EEPSQ

SEQ ID NO: 74 - protein, 117 aa, Artificial Sequence
  Synthetic heavy chain variable region of anti-c-Met antibody (AbF46 or huAbF46-H1)
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSS

SEQ ID NO: 75 - protein, 114 aa, Artificial Sequence
  Synthetic light chain variable region of anti-c-Met antibody (AbF46 or huAbF46-H1)
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKR

SEQ ID NO: 76 - DNA, 1416 bp, Artificial Sequence; identical to SEQ ID NO: 38
  Synthetic nucleotide sequence of heavy chain of anti-c-Met antibody (AbF46 or huAbF46-H1)

SEQ ID NO: 77 - DNA, 759 bp, Artificial Sequence; identical to SEQ ID NO: 39
  Synthetic nucleotide sequence of light chain of anti-c-Met antibody (AbF46 or huAbF46-H1)

SEQ ID NO: 78 - DNA, 4170 bp, Artificial Sequence
  Synthetic polynucleotide encoding c-Met protein
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 79 - protein, 444 aa, Artificial Sequence
  Synthetic SEMA domain of c-Met
LHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNG

SEQ ID NO: 80 - protein, 451 aa, Artificial Sequence
  Synthetic PSI-IPT domain of c-Met
YTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTG

SEQ ID NO: 81 - protein, 313 aa, Artificial Sequence
  Synthetic TyrKc domain of c-Met
VHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 82 - DNA, 1332 bp, Artificial Sequence
  Synthetic polynucleotide encoding SEMA domain of c-Met
ctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggc

SEQ ID NO: 83 - DNA, 1299 bp, Artificial Sequence
  Synthetic polynucleotide encoding PSI-IPT domain of c-Met
tacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacagga

SEQ ID NO: 84 - DNA, 939 bp, Artificial Sequence
  Synthetic polynucleotide encoding TyrKc domain of c-Met
gtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatca

SEQ ID NO: 85 - protein, 13 aa, Artificial Sequence
  Synthetic heavy chain CDR3 of anti-c-Met antibody
DNWFAYWGQGTLV

SEQ ID NO: 86 - protein, 10 aa, Artificial Sequence
  Synthetic light chain CDR3 of anti-c-Met antibody
LTFGAGTKLE

SEQ ID NO: 87 - protein, 117 aa, Artificial Sequence
  Synthetic heavy chain variable region of monoclonal antibody AbF46
EVKLVESGGGLVQPGGSLRLSCATSGFTFTDYYMSWVRQPPGKALEWLGFIRNKANGYTTEYSASVKGRFTISRDNSQSILYLQMDTLRAEDSATYYCARDNWFAYWGQGTLVTVSA

SEQ ID NO: 88 - protein, 114 aa, Artificial Sequence
  Synthetic light chain variable region of anti-c-Met antibody
DILMTQSPSSLTVSAGEKVTMSCKSSQSLLASGNQNNYLAWHQQKPGRSPKMLIIWASTRVSGVPDRFIGSGSGTDFTLTINSVQAEDLAVYYCQQSYSAPLTFGAGTKLELKR

SEQ ID NO: 89 - protein, 17 aa, Artificial Sequence
  Synthetic light chain CDR3 of anti-c-Met antibody
QQSYSAPLTFGAGTKLE

SEQ ID NO: 90 - protein, 117 aa, Artificial Sequence
  Synthetic heavy chain variable region of AT-VH1
EVKLVESGGGLVQPGGSLRLSCATSGFTFTDYYMSWVRQPPGKGLEWLGFIRNKANGYTTEYSASVKGRFTISRDNSKSTLYLQMNSLRAEDSATYYCARDNWFAYWGQGTLVTVSS

SEQ ID NO: 91 - protein, 117 aa, Artificial Sequence
  Synthetic heavy chain variable region of AT-VH2
EVKLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 92 - protein, 117 aa, Artificial Sequence
  Synthetic heavy chain variable region of AT-VH3
EVQLVESGGGLVQPGGSLRLSCATSGFTFTDYYMSWVRQPPGKGLEWLGFIRNKANGYTTEYSASVKGRFTISRDNSKSTLYLQMNSLRAEDTATYYCARDNWFAYWGQGTLVTVSS

SEQ ID NO: 93 - protein, 117 aa, Artificial Sequence
  Synthetic heavy chain variable region of AT-VH4
EVQLVESGGGLVQPGGSLRLSCATSGFTFTDYYMSWVRQPPGKGLEWLGFIRNKANGYTTEYSASVKGRFTISRDNSKNTLYLQMNSLRAEDTATYYCARDNWFAYWGQGTLVTVSS

SEQ ID NO: 94 - protein, 117 aa, Artificial Sequence
  Synthetic heavy chain variable region of AT-VH5
EVQLVESGGGLVQPGGSLRLSCATSGFTFTDYYMSWVRQPPGKGLEWLGFIRNKANGYTTEYSASVKGRFTISRDNSKNTLYLQMNSLRAEDTAVYYCARDNWFAYWGQGTLVTVSS

SEQ ID NO: 95 - protein, 114 aa, Artificial Sequence
  Synthetic light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKR

SEQ ID NO: 96 - protein, 113 aa, Artificial Sequence
  Synthetic light chain variable region of AT-Vk1
DILMTQSPSSLTASVGDRVTMTCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 97 - protein, 113 aa, Artificial Sequence
  Synthetic light chain variable region of AT-Vk2
DILMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 98 - protein, 113 aa, Artificial Sequence
  Synthetic light chain variable region of AT-Vk3
DIQMTQSPSSLSASVGDRVTITCKSSQSLLASGNQNNYLAWHQQKPGKAPKMLIIWASTRVSGVPDRFIGSGSGTDFTLTISSLQAEDVAVYYCQQSYSAPLTFGQGTKLEIK

SEQ ID NO: 99 - protein, 113 aa, Artificial Sequence
  Synthetic light chain variable region of AT-Vk4
DIQMTQSPSSLSASVGDRVTITCKSSQSLLASGNQNNYLAWHQQKPGKAPKMLIIWASTRVSGVPDRFSGSGSGTDFTLTISSLQAEDVAVYYCQQSYSAPLTFGQGTKLEIK

SEQ ID NO: 100 - protein, 13 aa, Artificial Sequence
  Synthetic modified hinge region(U7-HC6)
EPSCDKHCCPPCP

SEQ ID NO: 101 - protein, 13 aa, Artificial Sequence
  Synthetic modified hinge region(U6-HC7)
EPKSCDCHCPPCP

SEQ ID NO: 102 - protein, 12 aa, Artificial Sequence
  Synthetic modified hinge region(U3-HC9)
ERKCCVECPPCP

SEQ ID NO: 103 - protein, 14 aa, Artificial Sequence
  Synthetic modified hinge region(U6-HC8)
EPRDCGCKPCPPCP

SEQ ID NO: 104 - protein, 13 aa, Artificial Sequence
  Synthetic modified hinge region(U8-HC5)
EKCDKTHTCPPCP

SEQ ID NO: 105 - protein, 15 aa, Artificial Sequence
  Synthetic human hinge region
EPKSCDKTHTCPPCP

SEQ ID NO: 106 - protein, 17 aa, Artificial Sequence
  Synthetic CDR-L1 of antibody L3-11Y
KSSQSLLAWGNQNNYLA

SEQ ID NO: 107 - protein, 114 aa, Artificial Sequence
  Synthetic amino acid sequence of light chain variable region of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKR

SEQ ID NO: 108 - protein, 220 aa, Artificial Sequence
  Synthetic amino acid sequence of light chain of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 5 aa, Artificial Sequence
  Synthetic CDR-H1 of anti-EGFR scFv
NYDMS

SEQ ID NO: 110 - protein, 17 aa, Artificial Sequence
  Synthetic CDR-H2 of anti-EGFR scFv
GISHSSGSKYYADSVKG

SEQ ID NO: 111 - protein, 13 aa, Artificial Sequence
  Synthetic CDR-H3 of anti-EGFR scFv
KDATPRPLKPFDY

SEQ ID NO: 112 - protein, 13 aa, Artificial Sequence
  Synthetic CDR-L1 of anti-EGFR scFv
TGSSSNIGNNDVS

SEQ ID NO: 113 - protein, 7 aa, Artificial Sequence
  Synthetic CDR-L2 of anti-EGFR scFv
DDNKRPS

SEQ ID NO: 114 - protein, 9 aa, Artificial Sequence
  Synthetic CDR-L3 of anti-EGFR scFv
GSWDASLNA

SEQ ID NO: 115 - protein, 121 aa, Artificial Sequence
  Synthetic Heavy chain variable region of anti-EGFR scFv
EVQLLESGGGLVQPGGSLRLSCAASGFTFSNYDMSWVRQAPGKGLEWVSGISHSSGSKYYADSVKGRFTISRDNSKNTLYLQMNSLRAEDTAVYYCAKDATPRPLKPFDYWGQGTLVTVSS

SEQ ID NO: 116 - protein, 111 aa, Artificial Sequence
  Synthetic Light chain variable region of anti-EGFR scFv
QSVLTQPPSASGTPGQRVTISCTGSSSNIGNNDVSWYQQLPGTAPKLLIYDDNKRPSGVPDRFSGSKSGTSASLAISGLRSEDEADYYCGSWDASLNAYVFGGGTKLTVLG

SEQ ID NO: 117 - protein, 121 aa, Artificial Sequence
  Synthetic heavy chain variable region of anti-EGFR antibody (modified)
EVQLLESGGGLVQPGGSLRLSCAASGFTFSNYDMSWVRQAPGKCLEWVSGISHSSGSKYYADSVKGRFTISRDNSKNTLYLQMNSLRAEDTAVYYCAKDATPRPLKPFDYWGQGTLVTVSS

SEQ ID NO: 118 - protein, 111 aa, Artificial Sequence
  Synthetic light chain variable region of anti-EGFR antibody (modified)
QSVLTQPPSASGTPGQRVTISCTGSSSNIGNNDVSWYQQLPGTAPKLLIYDDNKRPSGVPDRFSGSKSGTSASLAISGLRSEDEADYYCGSWDASLNAYVFGCGTKLTVLG

SEQ ID NO: 119 - DNA, 363 bp, Artificial Sequence
  Synthetic coding nucleotide sequence of heavy chain variable region of anti-EGFR antibody
gaggtgcagctgttggagtctgggggaggcttggtacagcctggggggtccctgagactctcctgtgcagcctctggattcacctttagcaattatgatatgagctgggtccgccaggctccagggaaggggctggagtgggtctcagggatctctcatagtagtggtagtaaatattacgctgattctgtaaaaggtcggttcaccatctccagagacaattccaagaacacgctgtatctgcaaatgaacagcctgagagccgaggacacggccgtgtattactgtgcgaaagatgctactccgcgtccgctgaagcctttcgactactggggccagggtacactggtcaccgtgagctca

SEQ ID NO: 120 - DNA, 333 bp, Artificial Sequence
  Synthetic coding nucleotide sequence of light chain variable region of anti-EGFR antibody
cagtctgtgctgactcagccaccctcagcgtctgggacccccgggcagagggtcaccatctcttgtactggctcttcatctaatattggcaataatgatgtctcctggtaccagcagctcccaggaacggcccccaaactcctcatctatgatgataataagcggccaagcggggtccctgaccgattctctggctccaaatctggcacctcagcctccctggccatcagtgggctccggtccgaggatgaggctgattattactgtggttcttgggatgctagcctgaatgcttatgtcttcggcggaggcaccaagctgacggtcctaggc

SEQ ID NO: 121 - protein, 120 aa, Artificial Sequence [sequence omitted]

SEQ ID NO: 122 - DNA, 360 bp, Artificial Sequence
  Synthetic DNA encoding heavy-chain variable region of anti-EGFR antibody
caggtgcagctggtgcagtctggggctgaggtgaagaagcctgggtcctcggtgaaggtctcctgcaaggcctctggtttcacattcactgactacaagatacactgggtgcgacaggcccctggacaagggctcgagtggatgggatatttcaaccctaacagcggttatagtacctacgcacagaagttccagggcagggtcaccattaccgcggacaaatccacgagcacagcctacatggagctgagcagcctgagatctgaggacacggccgtgtattactgtgcgagactatccccaggcggttactatgttatggatgcctggggccaagggaccaccgtgaccgtctcctca

SEQ ID NO: 123 - protein, 108 aa, Artificial Sequence
  Synthetic light-chain variable region of anti-EGFR antibody
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 124 - DNA, 324 bp, Artificial Sequence
  Synthetic DNA encoding light-chain variable region of anti-EGFR antibody
gatatccagatgacccagtctccatcctccctgtctgcatctgtcggagaccgggtcaccatcacctgccgggcaagtcagggcattaacaattacttaaattggtaccagcagaagccagggaaagcccctaagcgcctgatctataataccaacaacttgcagacaggcgtcccatcaaggttcagcggcagtggatccgggacagaattcactctcaccatcagcagcctgcagcctgaagattttgccacctattactgcttgcagcataatagttttcccacgtttggccagggcaccaagctcgagatcaagcgtacg

SEQ ID NO: 125 - protein, 120 aa, Artificial Sequence
  Synthetic modified heavy-chain variable region of anti-EGFR antibody
QVQLVQSGAEVKKPGSSVKVSCKASGFTFTDYKIHWVRQAPGQCLEWMGYINPNSGYSTYASKFQGRVTITADKSTSTAYMELSSLRSEDTAVYYCARLSPGGYYVMDAWGQGTTVTVSS

SEQ ID NO: 126 - protein, 108 aa, Artificial Sequence
  Synthetic modified light-chain variable region of anti-EGFR antibody
DIQMTQSPSSLSASVGDRVTITCRASQGINNYLNWYQQKPGKAPKLLIYNTNNLQTGVPSRFSGSGSGTEFTLTISSLQPEDEATYYCLQHNSFPTFGQCTKLEIKRT

SEQ ID NO: 127 - protein, 442 aa, Artificial Sequence
  Synthetic Heavy chain of anti-c-Met antibody B
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 215 aa, Artificial Sequence
  Synthetic Light chain of anti-c-Met antibody B
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGDC

SEQ ID NO: 129 - protein, 114 aa, Artificial Sequence
  Synthetic light chain variable region of anti c-Met antibody
DIQMTQSPSSLSASVGDRVTITCKSSQSLLASGNQNNYLAWYQQKPGKAPKMLIIWASTRVSGVPSRFSGSGSGTDFTLTISSLQPEDFATYYCQQSYSRPYTFGQGTKVEIKR